(19) United States
(12) Patent Application Publication
(10) Pub. No.: US 2008/0108932 A1
(43) Pub. Date: May 8, 2008

(54) MEMS filter module with multi-level filter traps

(76) Inventor: M. Steven Rodgers, Albuquerque, NM (US)

Correspondence Address: David W. Highet, VP & Chief IP Counsel;Becton, Dickinson and Company, 1 Becton Drive, MC 110, Franklin Lakes, NJ, 07417-1880, US

(21) Appl. No.: 11/281,274

(22) Filed: Nov. 17, 2005

Related U.S. Application Data
(60) Provisional application No. 60/711,090, filed on Aug. 24, 2005.

(51) Int. Cl. A61F 9/00; A61F 2/14; A61M 39/08

(52) U.S. Cl. 604/8; 210/321.72

(57) ABSTRACT

A MEMS flow module (340) includes a plurality of filtering sections (344). Each filtering section (344) is defined by a stack (342) of a plurality of layers (346, 348, 350, 352). Each filtering section (344) includes at least one filter trap (364, 368) at each of at least two different levels or elevations within the stack (342). This provides for an increased flow rate through the MEMS flow module (340).

### CROSS-REFERENCE TO RELATED APPLICATIONS

This patent application claims priority under 35 U.S.C. §119(e) to U.S. Provisional Patent Application Ser. No. 60/711,090, that was filed on Aug. 24, 2005, that is entitled “MEMS FILTER MODULE WITH MULTI-LEVEL FILTER TRAPS,” and the entire disclosure of which is hereby incorporated by reference in its entirety herein.

### FIELD OF THE INVENTION

The present invention generally relates to the field of filters and, more particularly to a MEMS filter with filter traps that exist at least at two different elevations to accommodate an increased flow rate through the MEMS filter.

### BACKGROUND OF THE INVENTION

High internal pressure within the eye can damage the optic nerve and lead to blindness. There are two primary chambers in the eye—an anterior chamber and a vitreous body that are generally separated by a lens. Aqueous humor exists within the anterior chamber, while vitreous humor exists in the vitreous body. Generally, an increase in the internal pressure within the eye is caused by more fluid being generated within the eye than is being discharged by the eye. The general consensus is that it is excess fluid within the anterior chamber of the eye that is the main contributor to an elevated intraocular pressure.

One proposed solution to addressing high internal pressure within the eye is to install an implant. Implants are typically directed through a wall of the patient's eye so as to fluidly connect the anterior chamber with an exterior location on the eye. There are a number of issues with implants of this type. One is the ability of the implant to respond to changes in the internal pressure within the eye in a manner that reduces the potential for damaging the optic nerve. Another is the ability of the implant to reduce the potential for bacteria and the like passing through the implant and into the interior of the patient's eye, for instance into the anterior chamber.

### BRIEF SUMMARY OF THE INVENTION

The present invention generally relates to a MEMS filter module that provides a filtering function somewhere between multiple pairs of adjacent MEMS layers. One particularly desirable application for this MEMS filter module is for use in an implant or a drainage device that is installable at least partially in a biological mass. For instance, this MEMS filter module may be used in a device that is installed in a human eye to treat glaucoma by relieving excess intraocular pressure.

A first aspect of the present invention is embodied by a MEMS filter module that may be used in any appropriate application. This MEMS filter module includes a first filtering section that includes a stack of a plurality of structurally interconnected layers. These layers are stacked in a first dimension, and each layer includes at least one flow port that extends completely through its corresponding layer. The first filtering section also includes first and second filter traps. All flow through each of the first and second filter traps is in a second dimension that is different from the first dimension, and each of the first and second filter traps provides a greater flow resistance than each individual flow port.

Various refinements exist of the features noted in relation to the first aspect of the present invention. Further features may also be incorporated in the first aspect of the present invention as well. These refinements and additional features may exist individually or in any combination. One or more adjacent pairs of layers in the stack may be the fabricated so as to be disposed in spaced relation and structurally interconnected in any appropriate manner (e.g., by a plurality of posts, columns, or other structural interconnects of any appropriate size, shape, and configuration that extend between an adjacent pair of layers). One or more adjacent pairs of layers in the stack also may be fabricated directly on each other, but where a cavity is retained between these layers to define a filter trap. In any case, at least one annular seal preferably exists between each adjacent pair of layers within this stack to reduce the potential for a flow exiting the MEMS filter module between an adjacent pair of layers. “Annular” in relation to this seal (or any other structure characterized herein as being annular) merely means that the relevant structure extends a full 360° about a reference axis. Representative annular configurations include circular, rectangular, square, elliptical, or the like. In any case, such an annular seal is disposed about the first filtering section. Multiple first filtering sections may be disposed inwardly of such annular seals as well.

The various layers in the stack may be of any appropriate material (e.g., polysilicon). Each layer in the stack may be of the same material, or two or more layers may be formed from different materials (e.g., polysilicon and silicon nitride). Any appropriate number of layers may be used in the stack, although there will typically be at least three separate layers. In one embodiment, the stack includes first, second, and third layers, where the second layer is located between the first and third layers, where the first filter trap is associated with a flow path somewhere between the first and second layers, and where the second filter trap is associated with a flow path somewhere between the second and third layers.

Each of the various layers of the stack may be of any appropriate thickness. In one embodiment, each layer has a maximum thickness of about 10 microns. Another embodiment has each layer with a maximum thickness being within a range of about 1 micron to about 3 microns. Surface micromachining is the preferred technology for fabricating the MEMS filter module. Each layer of the MEMS filter module could then be associated with a different fabrication level. “Fabrication level” corresponds with what may be formed by a deposition of a structural material before having to form any overlying layer of a sacrificial material (e.g., from a single deposition of a structural layer or film). Having the first filter trap being defined somewhere between a first pair of adjacent layers and the second filter trap being defined somewhere between a second pair of adjacent layers thereby disposes the first and second filter traps at different elevations within the stack.

The flow through the first and second filter traps is in a dimension that is different than the dimension in which the thickness of the stack extends. In one embodiment, the flow through the first and second filter traps is within a dimension that is orthogonal to the thickness dimension of the stack. Stated another way, the flow through the first and second filter traps may be characterized as being perpendicular to the flow through the various flow ports of the stack. Although the flow through the first and second filter traps is within a common dimension, this does not limit the flow to being in the same direction. For instance, the flow through the first and second filter traps may be in what may be characterized as opposite directions. One example is where the flow through the first filter trap is in an inwardly direction relative to a reference axis that extends through the thickness of the stack, and where the flow through the second filter trap is in an outwardly direction relative to this same reference axis. However, the flow through the first and second filter traps may be in a common direction as well (e.g., the flow through the first and second filter traps could be inwardly relative to a reference axis that extends through the thickness of the stack; the flow through the first and second filter traps could be outwardly relative to a reference axis that extends through the thickness of the stack).

The first and second filter traps may be of any appropriate size, shape, and/or configuration. In one embodiment, the first and second filter traps each have a height dimension of no more than about 4 microns, where the “height dimension” is that which is perpendicular with the direction of the flow through the particular filter trap. One representative way of defining one or both of the first and second filter traps is to have a filtering wall that extends from one layer toward an adjacent layer, where this filtering wall and the adjacent layer are spaced. Such a filtering wall may be of any appropriate annular configuration, or may be in the form of a plurality of filtering wall segments that are appropriately spaced from each other. In any case, it should be appreciated there could be spacings of at least two different magnitudes between adjacent layers when defining a filter trap using a filtering wall that extends from one layer and that terminates prior to reaching an adjacent layer. Consider the case where a filtering wall extends from a second layer and terminates prior to reaching a first layer. Here, a distal end of the filtering wall and the first layer could be separated by a spacing of a first magnitude, while the first and second layers may be separated by a spacing of a second magnitude that is larger than the first magnitude.

Another representative way of defining one or both of the first and second filter traps is by the space between an adjacent pair of layers. The spacing between an adjacent pair of layers that defines a filter trap is subject to a number of characterizations. One is that a maximum spacing between a pair of adjacent layers defines its corresponding filter trap. Another is that a common, constant spacing exists between a pair of layers and defines its corresponding filter trap. Yet another is that substantially planar, opposing surfaces of a common size from an adjacent pair of layers collectively define a corresponding filter trap.

The first and second filtering traps may be of the same configuration or may be of different configurations. For instance, the first and second filtering traps each could be defined by a separate filtering wall of the above-noted type (of the same or a different size/shape/configuration), or each could be defined by the space between an adjacent pair of layers. One of the first and second filtering traps could be defined by a filtering wall of the above-noted type, while the other of the first and second filtering traps could be defined by the space between adjacent pair of layers. The first and second filtering traps could provide the same flow resistance, or the first and second filtering traps could provide a different flow resistance. For instance, the length of the first and second filtering traps could be the same or different, the cross-sectional area of the first and second filtering traps, taken perpendicularly to direction of the flow therethrough, could be the same or different, or any combination thereof.

A number of particular embodiments in accordance with the first aspect of the present invention will now be addressed. Unless otherwise noted, each of these embodiments will provide a filtering function, regardless of the direction of the flow therethrough. These embodiments address a number of permutations regarding, for instance, the numbers of layers, the number of flow ports, the arrangement of flow ports, filter trap configurations, the arrangement of filtering traps, and the like. At least some of these embodiments will address “Group I flow ports” and “Group II flow ports.” Each Group I flow port shares at least one common characteristic, while each Group II flow port shares at least one common characteristic. However, all characteristics of each of the various Group I flow ports need not be the same from layer to layer. Similarly, all characteristics of each of the various Group II flow ports need not be the same from layer to layer. In one embodiment, all flow between any Group I flow port and any Group II flow port must pass through at least one filtering trap, a flow through a Group II flow port in one layer may pass through a Group II flow port in an adjacent layer without passing through any filtering trap, and a flow through a Group I flow port in one layer may pass through a Group I flow port in an adjacent layer without passing through any filtering trap. In another embodiment, the Group I flow ports are disposed within a first region, while the Group II flow ports are disposed in a second region that is disposed about the first region. In at least some embodiments, the Group I flow ports are associated with one side of the various filter traps of the MEMS filter module (e.g., an inlet side or an outlet side), while the Group II flow ports are associated with the opposite side of the various filter traps of the MEMS filter module. In a second instance, if the Group I flow ports are associated with the inlet side of the various filter traps of the MEMS filter module, the Group II flow ports would be associated with the outlet side of the various filter traps of the MEMS filter module, and vice versa.

A first embodiment of a MEMS filter module in accordance with the first aspect has a first filtering section with first and second end layers (the two opposing extremes of the stack), at least one intermediate layer, and a plurality of filter traps that includes the first and second filter traps. The first end layer includes at least one Group I flow port, but does not include any Group II flow ports. The second end layer includes at least one Group II flow port, but does not include any Group I flow ports. At least one intermediate layer, and more preferably each intermediate layer, includes at least one Group I flow port and at least one Group II flow port. The various Group I and Group II flow ports, as well as the various filter traps, are arranged such that all flow between any Group I flow port of the first end layer and any Group II flow port of the second end layer is required to flow through at least one filter trap, and including flowing through only a single filter trap.

A second embodiment of a MEMS filter module in accordance with the first aspect has a first filtering section with first, second, and third layers, as well as a plurality of filter traps that includes the first and second filter traps. The second layer is located between the first and third layers. Each of the first, second, and third layers includes at least one Group I flow port, or at least one Group II flow port, or at least one Group I flow port and at least one Group II flow port. Each Group I flow port and each Group II flow port provides a smaller flow resistance than any of the plurality of filter traps. The first and second layers each include a first Group I flow port, the second and third layers each include a first Group II flow port, the first layer does not include any Group II flow port, and the third layer does not include any Group I flow port.

A third embodiment of a MEMS filter module in accordance with the first aspect has a first filtering section with first and second end layers (the two opposing extremes of the stack), at least one intermediate layer, and a plurality of filter traps that includes the first and second filter traps. The first and second end layers each include either at least one Group I flow port and no Group II flow ports, or at least one Group II flow port and no Group I flow ports. At least one intermediate layer, and more preferably each intermediate layer, includes at least one Group I flow port and at least one Group II flow port. The various Group I and Group II flow ports, as well as the various filter traps, are arranged such that all flow between any flow port of the first end layer and any flow port of the second end layer is required to pass through at least one of the plurality of filter traps at each of two different locations or elevations in the first dimension. In one embodiment, the flow through such a filter trap at one location in the first dimension is in one direction while the flow through such a filter trap at any different location in the first dimension is in a different direction, and including being in an at least generally opposite direction.

A fourth embodiment of a MEMS filter module in accordance with the first aspect has a first filtering section with a first pair of adjacent layers and a second pair of adjacent layers. Each layer of the first pair of adjacent layers and each layer of the second pair includes at least one Group I flow port, or at least one Group II flow port, or at least one Group I flow port and at least one Group II flow port. Each Group I flow port and each Group II flow port provides a smaller flow resistance than the first and second filter traps. An at least substantially constant spacing exists between each layer of the first pair of adjacent layers and defines the first filter trap. Similarly, an at least substantially constant spacing exists between each layer of the second pair of adjacent layers and defines the second filter trap. The spacing between the layers of the first pair of adjacent layers may be the same or different as the spacing between the layers of the second pair of adjacent layers. The first and second filter traps may be of the same length or a different length as well. Therefore, the flow resistance provided by the first and second filter traps may be the same or different.

A fifth embodiment of a MEMS filter module in accordance with the first aspect has a first filtering section with first and second end layers (the two opposing extremes of the stack), at least one intermediate layer, and a plurality of filter traps that includes the first and second filter traps. Each of the first and second end layers, as well as at least one of the intermediate layers and thereby including each intermediate layer, includes at least one Group I flow port, or at least one Group II flow port, or at least one Group I flow port and at least one Group II flow port. Each Group I flow port and each Group II flow port provides a smaller flow resistance than the first and second filter traps. An at least substantially constant spacing exists between adjacent pair of layers, and this spacing defines a corresponding filter trap. The first end layer includes at least one Group II flow port and does not include any Group I flow ports. Each intermediate layer includes a plurality of Group II flow ports and at least one first Group I flow port. The second end layer includes at least one Group I flow port and does not include any Group II flow ports.

Each Group II flow port of the first layer may be axially aligned with a corresponding Group II flow port in each of the various intermediate layers in the above-noted fifth embodiment, while each Group I flow port of each intermediate layer may be axially aligned with a corresponding Group I flow port in the second end layer. Further in this regard, the size of the various Group I and Group II flow ports each may progressively decrease from layer to layer proceeding in the direction of the second end layer. Alternatively, each Group I flow port may be of the same size throughout the various layers, while each Group II flow port may be of the same size throughout the various layers as well.

The constant spacing between adjacent layers in the case of the fifth embodiment may be the same or different amongst the various pairs. That is, the spacing between a first pair of adjacent layers may be of a first magnitude, while the spacing between a second pair of adjacent layers may also be of the first magnitude or may be any different second magnitude. The various filter traps may be of the same length or a different length as well. Therefore, the flow resistance provided by the various filter traps may be the same or different.

Each of the MEMS filter modules described herein may be used in combination with a conduit to define a drainage device or an implant that is at least partially installable in a biological mass. In this regard, the conduit may include a flow path that is adapted to fluidly interconnect a first body region and any appropriate drainage location, and at least one MEMS filter module may be disposed within this flow path. In one embodiment, at least one housing is used to establish an interconnection or interface between the conduit and the MEMS filter module. For instance, the housing may be at least partially disposed within the conduit, and the MEMS filter module may interface with the housing. Although any appropriate application is contemplated, in one embodiment the drainage device is installable in a human eye to fluidly interconnect with an anterior chamber of the human eye for purposes of regulating intraocular pressure.

A second aspect of the present invention is embodied by a MEMS filter module, which may be used in any appropriate application. This MEMS filter module includes a first filtering section, which in turn includes a stack of at least three structurally interconnected layers. Each layer in the first filtering section includes at least one flow port that extends completely through its thicknesses. At least one filter trap exists between each adjacent pair of layers in the first filtering section. Therefore, at least one filter trap exists at each of multiple levels or elevations within the stack. The various flow ports and filter traps are arranged such that a flow must progress through at least one filter trap at a first elevation within the stack, and thereafter through at least one filter trap at a second elevation within the stack in order to completely progress through the first filtering section.

Various refinements exist of the features noted in relation to the second aspect of the present invention. Further features may also be incorporated in the second aspect of the present invention as well. These refinements and additional features may exist individually or in any combination. Initially, the various features discussed above in relation to the first aspect may be used by this second aspect, individually or in any combination. The filter traps may be of any appropriate size, shape, and/or configuration. In one embodiment, all filter traps are defined in the same general manner. In another embodiment, a filter trap between a first pair of adjacent layers is defined in one manner (e.g., by a filter wall that protrudes from one layer toward an adjacent layer), while a filter trap between a second pair of adjacent layers is defined in another manner (e.g., by a spacing between the adjacent layers of the second pair).

A third aspect of the present invention is embodied by a MEMS filter module, which may be used in any appropriate application. This MEMS filter module includes a first filtering section, which in turn includes a stack of a plurality of structurally interconnected layers. Each layer in the first filtering section includes at least one flow port that extends completely through its thicknesses. Generally, the spacing between at least one adjacent pair of layers defines a filter trap in the case of the third aspect.

Various refinements exist of the features noted in relation to the third aspect of the present invention. Further features may also be incorporated in the third aspect of the present invention as well. These refinements and additional features may exist individually or in any combination. Initially, the various features discussed above in relation to the first aspect may be used by this third aspect, individually or in any combination. The MEMS filter module may be fabricated by surface micromachining in a manner that reduces the number of masking operations (e.g., by simultaneously forming apertures through multiple layers).

In one embodiment, the spacing between each adjacent pair of layers in the first filtering section defines a filter trap. The spacing between an adjacent pair of layers that defines a filter trap is subject to a number of characterizations. One is at a maximum spacing between a pair of adjacent layers defines its corresponding filter trap. Another is that a common, constant spacing exists between a pair of layers and defines its corresponding filter trap (e.g., a given filter trap need not defined by a structure that protrudes from one layer toward another layer). Another is that substantially planar, opposing surfaces of a common size from an adjacent pair of layers collectively define its corresponding filter trap.

### BRIEF DESCRIPTION OF THE SEVERAL VIEWS OF THE DRAWING

FIG. 1 is a side view of a plurality of layers that may be used by one embodiment of a surface micromachining fabrication technique.

FIG. 2 is a perspective view of one embodiment of a MEMS filtering section, which has filter traps at three different levels.

FIG. 3A is a perspective view of one embodiment of an MEMS filter module having filter traps disposed at multiple levels.

FIG. 3B is a perspective view of one filtering section from the MEMS filter module of FIG. 3A, which has filter traps at three different levels.

FIG. 3C is a cross-sectional view of the filtering section of FIG. 3B.

FIG. 3D is a cutaway, exploded, perspective view of opposing surfaces of first and second layers of the MEMS filter module of FIG. 3A.

FIG. 3E is a cutaway, exploded, perspective view of opposing surfaces of second and third layers of the MEMS filter module of FIG. 3A.

FIG. 3F is a cutaway, exploded, perspective view of opposing surfaces of third and fourth layers of the MEMS filter module of FIG. 3A.

FIG. 4A is a perspective view of another embodiment of a MEMS filtering section, which has filter traps at four different levels.

FIG. 4B is a cross-sectional view of the MEMS filtering section of FIG. 4A.

FIG. 5A is a perspective view of another embodiment of a MEMS filtering section, which has filter traps at four different levels, and which requires a flow to pass through at least one filter trap at each of two different levels.

FIG. 5B is a cross-sectional view of the MEMS filtering section of FIG. 5A.

FIG. 5C is a cross-sectional view of an alternative configuration of the MEMS filtering section of FIG. 5A.

FIG. 6A is a perspective view of another embodiment of a MEMS filtering section, which has filter traps at four different levels, and where each filter trap is defined by a space between adjacent layers.

FIG. 6B is a cross-sectional view of the MEMS filtering section of FIG. 6A.

FIG. 6C is a perspective view of a patterned first end layer of the MEMS filtering section of Figure of 6A.

FIG. 6D is a perspective view of a patterned first sacrificial layer on the first end layer of FIG. 6C.

FIG. 6E is a perspective view of a patterned second layer on the patterned first sacrificial layer of FIG. 6D.

FIG. 7A is a cross-sectional view of another embodiment of a MEMS filtering section, which has filter traps at four different levels, and where each filter trap is defined by a space between adjacent layers.

FIG. 7B is a perspective, cross-sectional view of the lower four structural layers of the MEMS filtering section of FIG. 7A, after a single patterning operation to define a Group I flow port in each of these layers.

FIG. 7C is a perspective, cross-sectional view of the lower four structural layers from FIG. 7B, after the deposition of a sacrificial layer and a subsequent single patterning operation to define a structural interconnect aperture between the various layers.

FIG. 7D is a perspective, cross-sectional view, after depositing an end layer onto the configuration of FIG. 7C.

FIG. 7E is a perspective, cross-sectional view, after a single patterning operation to define a plurality of Group II flow ports in the upper four structural layers of the MEMS filtering section of FIG. 7A.

FIG. 7F is a perspective, cross-sectional view of a variation of the MEMS filtering section of FIG. 7A.

FIG. 8A is an exploded, perspective view of one embodiment of a flow assembly that uses a MEMS flow module.

FIG. 8B is a perspective view of the flow assembly of FIG. 8A in an assembled condition.

FIG. 9A is an exploded, perspective of another embodiment of a flow assembly that uses a MEMS flow module.

FIG. 9B is a perspective view of the flow assembly of FIG. 9A in an assembled condition.

FIG. 10A is an exploded, perspective of another embodiment of a flow assembly that uses a MEMS flow module.

FIG. 10B is a perspective view of the flow assembly of FIG. 10A in an assembled condition.

FIG. 11A is a schematic of one embodiment of a glaucoma drainage device that may use any of the MEMS filter modules described herein.

FIG. 11B is a cross-sectional view of one embodiment of a glaucoma drainage device that is used to relieve pressure within the anterior chamber of the eye, and that may utilize any of the MEMS filter modules described herein.

### DETAILED DESCRIPTION OF THE INVENTION

The present invention will now be described in relation to the accompanying drawings that at least assist in illustrating its various pertinent features. Generally, the devices described herein are microfabricated. There are a number of microfabrication technologies that are commonly characterized as “micromachining,” including without limitation LIGA (Lithographie, Galvonoformung, Abformung), SLIGA (sacrificial LIGA), bulk micromachining, surface micromachining, micro electrodischarge machining (EDM), laser micromachining, 3-D stereolithography, and other techniques. Hereafter, the term “MEMS device,” “microfabricated device,” or the like means any such device that is fabricated using a technology that allows realization of a feature size of 10 microns or less. Any appropriate microfabrication technology or combination of microfabrication technologies may be used to fabricate the various devices to be described herein.

Surface micromachining is currently the preferred fabrication technique for the various devices to be described herein. One particularly desirable surface micromachining technique is described in U.S. Pat. No. 6,082,208, that issued Jul. 4, 2000, that is entitled “Method For Fabricating Five-Level Microelectromechanical Structures and Microelectromechanical Transmission Formed,” and the entire disclosure of which is incorporated by reference in its entirety herein. Surface micromachining generally entails depositing alternate layers of structural material and sacrificial material using an appropriate substrate (e.g., a silicon wafer) which functions as the foundation for the resulting microstructure. Various patterning operations (collectively including masking, etching, and mask removal operations) may be executed on one or more of these layers before the next layer is deposited so as to define the desired microstructure. After the microstructure has been defined in this general manner, all or a portion of the various sacrificial layers are removed by exposing the microstructure and the various sacrificial layers to one or more etchants. This is commonly called “releasing” the microstructure.

The term “sacrificial layer” as used herein means any layer or portion thereof of any surface micromachined microstructure that is used to fabricate the microstructure, but which does not generally exist in the final configuration (e.g., sacrificial material may be encased by a structural material at one or more locations for one or more purposes, and as a result this encased sacrificial material is not removed by the release). Exemplary materials for the sacrificial layers described herein include undoped silicon dioxide or silicon oxide, and doped silicon dioxide or silicon oxide (“doped” indicating that additional elemental materials are added to the film during or after deposition). The term “structural layer” as used herein means any other layer or portion thereof of a surface micromachined microstructure other than a sacrificial layer and a substrate on which the microstructure is being fabricated. Exemplary materials for the structural layers described herein include doped or undoped polysilicon and doped or undoped silicon. Exemplary materials for the substrates described herein include silicon. The various layers described herein may be formed/deposited by techniques such as chemical vapor deposition (CVD) and including low-pressure CVD (LPCVD), atmospheric-pressure CVD (APCVD), and plasma-enhanced CVD (PECVD), thermal oxidation processes, and physical vapor deposition (PVD) and including evaporative PVD and sputtering PVD, as examples.

In more general terms, surface micromachining can be done with any suitable system of a substrate, sacrificial film(s) or layer(s) and structural film(s) or layer(s). Many substrate materials may be used in surface micromachining operations, although the tendency is to use silicon wafers because of their ubiquitous presence and availability. The substrate is essentially a foundation on which the microstructures are fabricated. This foundation material must be stable to the processes that are being used to define the microstructure(s) and cannot adversely affect the processing of the sacrificial/structural films that are being used to define the microstructure(s). With regard to the sacrificial and structural films, the primary differentiating factor is a selectivity difference between the sacrificial and structural films to the desired/required release etchant(s). This selectivity ratio may be on the order of about 10:1, and is more preferably several hundred to one or much greater, with an infinite selectivity ratio being most preferred. Examples of such a sacrificial film/structural film system include: various silicon oxides/various forms of silicon; poly germanium/poly germanium-silicon; various polymeric films/various metal films (e.g., photoresist/aluminum); various metals/various metals (e.g., aluminum/nickel); polysilicon/silicon carbide; silicone dioxide/polysilicon (i.e., using a different release etchant like potassium hydroxide, for example). Examples of release etchants for silicon dioxide and silicon oxide sacrificial materials are typically hydrofluoric (HF) acid based (e.g., concentrated HF acid, which is actually 49 wt % HF acid and 51 wt % water; concentrated HF acid further diluted with water; buffered HF acid (HF acid and ammonium fluoride)).

The microfabrication technology described in the above-noted '208 Patent uses a plurality of alternating structural layers (e.g., polysilicon and therefore referred to as “P” layers herein) and sacrificial layers (e.g., silicon dioxide, and therefore referred to as “S” layers herein). The nomenclature that is commonly used to describe the various layers in the microfabrication technology described in the above-noted '208 Patent will also be used herein.

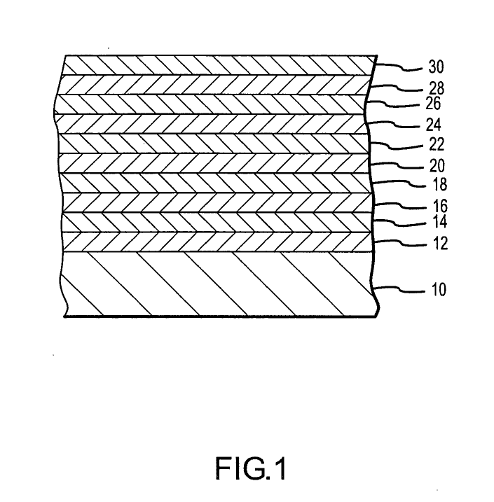

FIG. 1 generally illustrates one embodiment of layers on a substrate 10 that is appropriate for surface micromachining and in accordance with the nomenclature commonly associated with the '208 Patent. Each of these layers will typically have a thickness of no more than about 10 microns, and more typically a thickness within a range of about 1 micron to about 3 microns. Progressing away from the substrate 10, the various layers are: a dielectric layer 12 (there may be an intermediate oxide layer between the dielectric layer 12 and the substrate 10 as well, which is not shown); a P₀ layer 14 (a first fabrication level); an S₁ layer 16; a P₁ layer 18 (a second fabrication level); an S₂ layer 20; a P₂ layer 22 (a third fabrication level); an S₃ layer 24; a P₃ layer 26 (a fourth fabrication level); an S₄ layer 28; and a P₄ layer 30 (a fifth fabrication level). In some cases, the S₂ layer 20 may be removed before the release such that the P₂ layer 22 is deposited directly on the P₁ layer 18, and such will hereafter be referred to as a P₁/P₂ layer. It should also be appreciated that one or more other layers may be deposited on the P₄ layer 30 after the formation thereof and prior to the release, where the entirety of the S₁ layer 16, S₂ layer 20, S₃ layer 24, and S₄ layer 28 may be removed (although portions of one or more of these layers may be retained for one or more purposes if properly encased so as to be protected from the release etchant). It should also be appreciated that adjacent structural layers may be structurally interconnected by forming cuts or apertures through the entire thickness of a particular sacrificial layer before depositing the next structural layer. In this case, the structural material will not only be deposited on the upper surface of the particular sacrificial layer, but will be deposited in these cuts or apertures as well (and will thereby interconnect a pair of adjacent, spaced, structural layers).

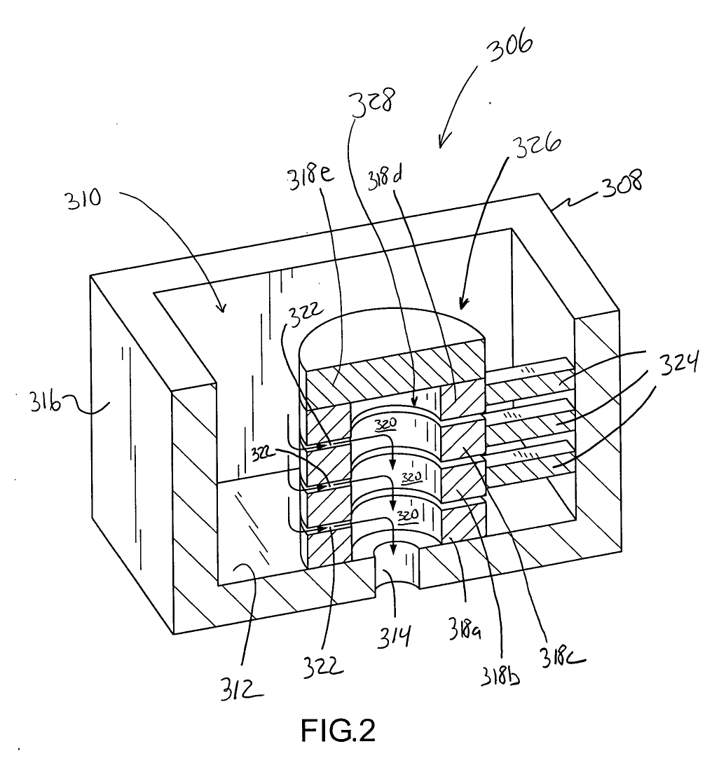

A schematic of one embodiment of a MEMS filtering section having filter traps disposed at multiple levels is illustrated in FIG. 2 and is identified by reference 306. The MEMS filtering section 306 generally includes a container 308 having an open end 310, an oppositely disposed closed end 312 having a flow port 314, and an annular sidewall 316 that extends from the closed end 312. “Annular” in relation to the sidewall 316, as well as any other structure described as “annular” herein, simply means that the relevant structure extends a full 360° about a reference point or axis, and does not limit is structure to being circular. Representative annular configurations include circular, square, rectangular, elliptical, and the like.

A flow through the MEMS filtering section 306 is filtered using a stack 326 of a plurality of plates or layers 318a-e. The stack 326 is disposed within the hollow interior of the container 308. Each of the layers 318a-d of the stack 326 includes a flow port 320. Adjacently disposed flow ports 320 within the stack 326 fluidly communicate with each other and collectively define an interior chamber 328. The flow port 320 through the layer 318a fluidly communicates with the flow port 314 through the closed end 312 of the container 308.

Each adjacent pair of layers 318a-d is disposed in spaced relation to define a filter trap 322. In this regard, the layer 318a is disposed directly on the closed end 312 of the container 308 to close one end of the interior chamber 328, while the layers 318b-d are disposed in the desired position by a support 324 that extends from the sidewall 316 of the container 308. However, the layer 318a could also be supported in spaced relation to the closed end 312 (not shown). The layer 318e is disposed directly on an end of the layer 318d to “seal” an opposite end of the interior chamber 328. Although each filter trap 322 is illustrated as being of the same height and length, such need not be the case. Each filter trap 322 may be of any appropriate height and length. Since the layers 318a-d each occupy different levels or elevations within the stack 326, each filter trap 322 likewise occupies a different level or elevation within the stack 326.

Fluid that enters the hollow interior of the container 308 (more specifically the space between the annular sidewall 316 and the stack 326) may be discharged through the flow port 314 on the closed end 312 of the container 308 by passing through any one of the filter traps 322. Similarly, any fluid that enters the interior chamber 328 of the stack 326 through the flow port 314 may be discharged into the hollow interior of the container 308 by passing through any one of the filter traps 322. Providing multiple filter traps 322 at multiple levels within the stack 326 allows for increased flow rate through the MEMS filtering section 306.

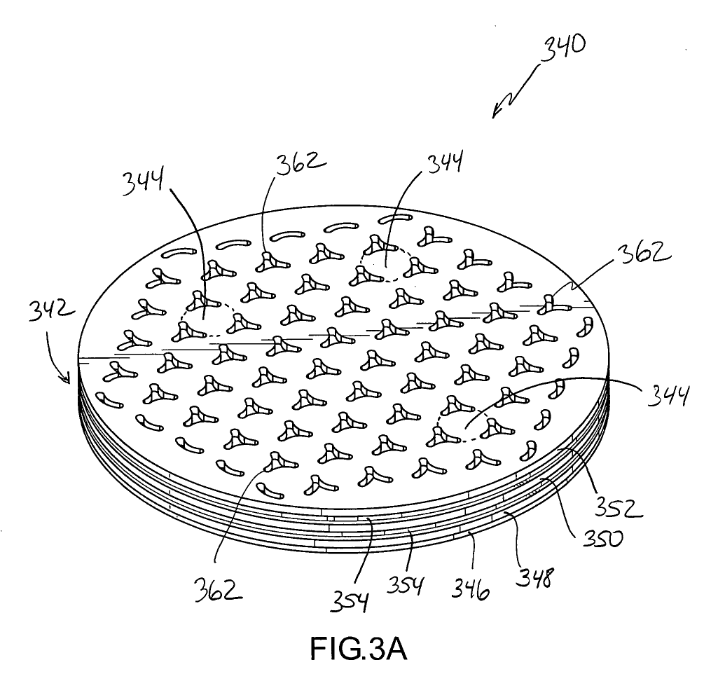

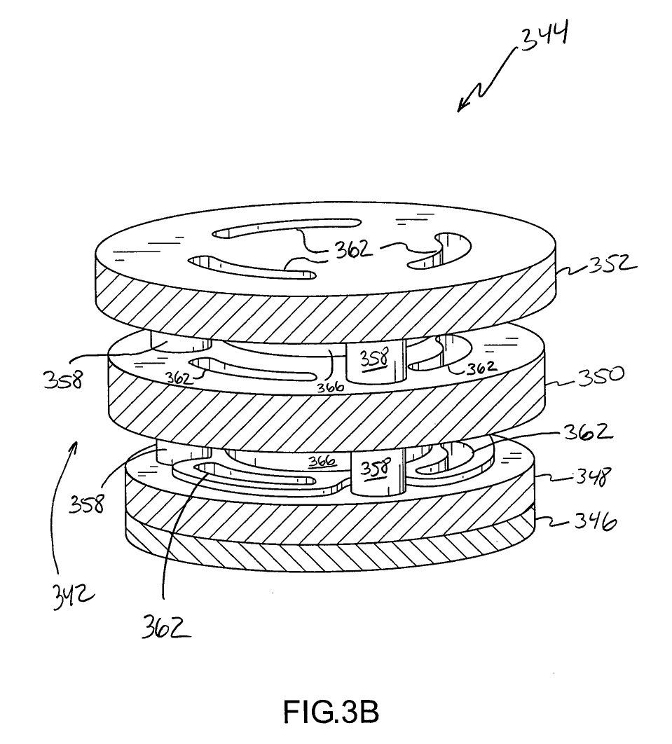

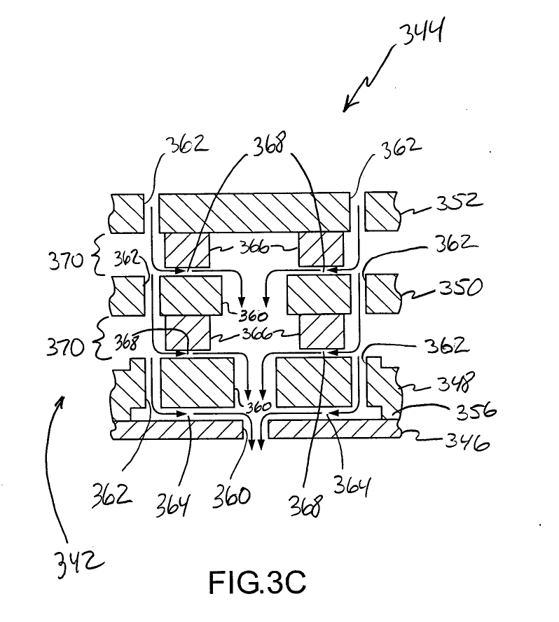

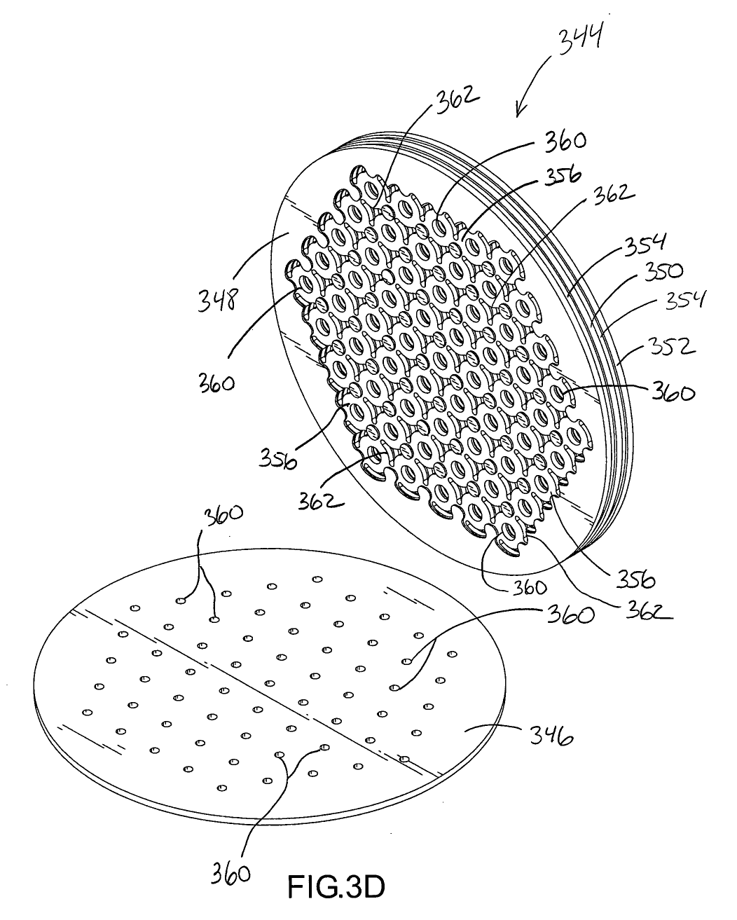

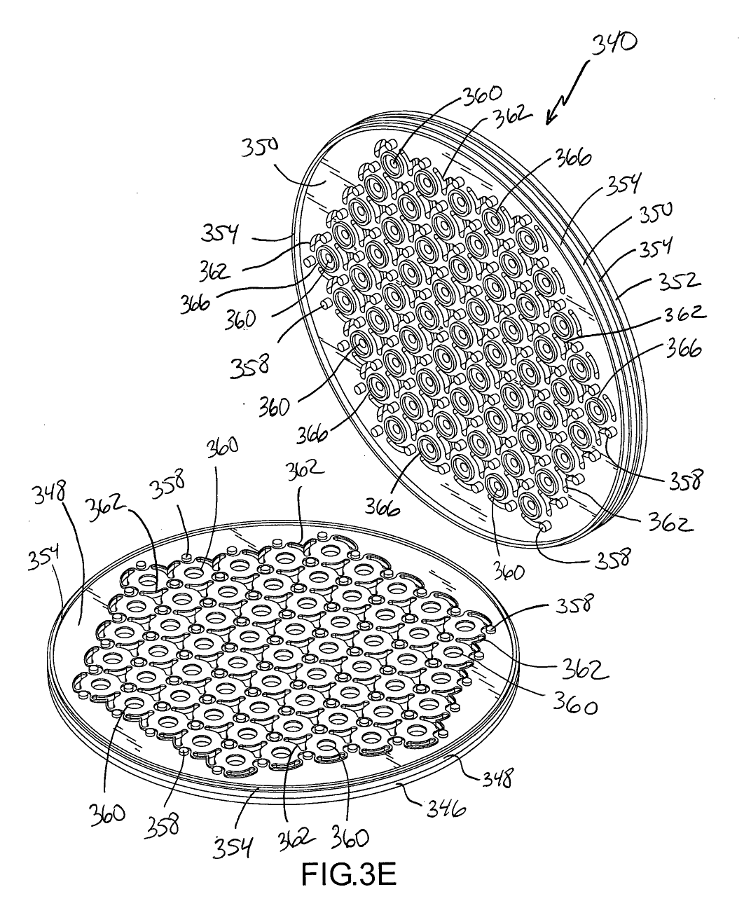

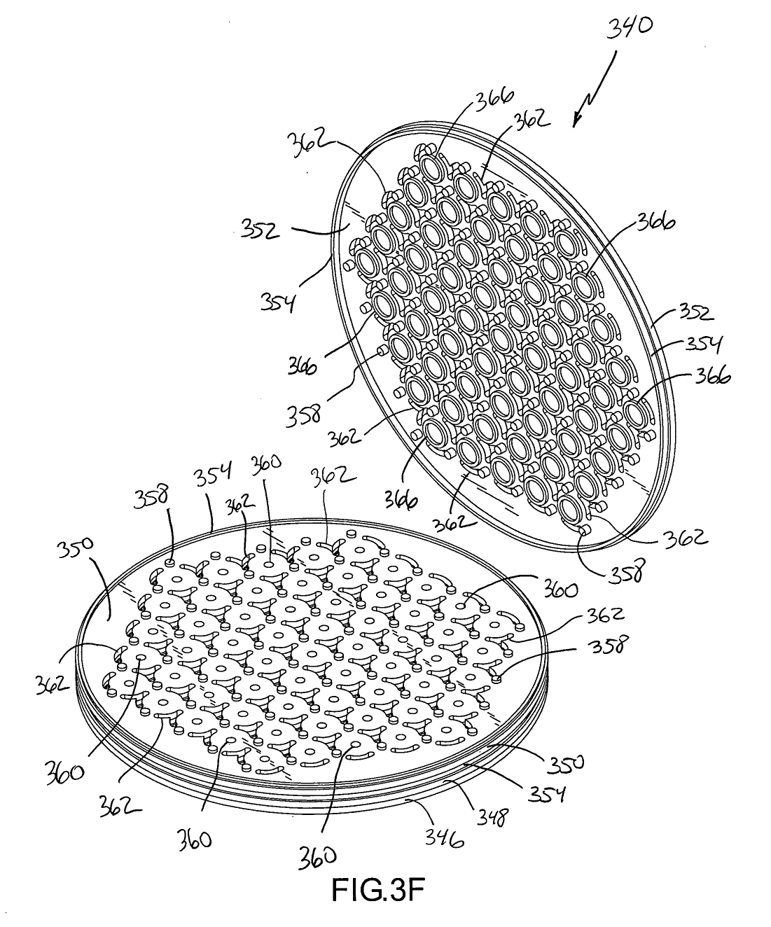

An embodiment of a MEMS filter module (more generally a MEM flow module—a MEMS device that accommodates a flow therethrough) that includes a plurality of filter traps at each of a plurality of levels is illustrated in FIGS. 3A-F and is identified by reference numeral 340. The cutaway view of FIG. 3D is taken immediately adjacent to the first end layer 346, with the remainder of the MEMS filter module 340 being pivoted away. The cutaway view of FIG. 3E is taken through the filter traps 368 between the second layer 348 in the third layer 350, and with the third layer 350 and structures interconnected therewith being pivoted away from the second layer 348. Finally, the cutaway view of FIG. 3F is taken through the filter traps 368 between the third layer 350 and the fourth layer 352, with the fourth layer 352 and structures interconnected therewith being pivoted away from the third layer 350. Cross-hatching is not included on the exposed ends of the structural interconnects 358 or on the exposed ends of the annular sealing walls 354 in FIGS. 3D-F, which extend between various adjacent layers as will be discussed in more detail below.

The MEMS filter module 340 is in the form of a stack 342 of a plurality of layers 346 (e.g., P₁ layer 18), 348 (e.g., P₂ layer 22), 350 (e.g., P₃ layer 26), and 352 (e.g., P₄ layer 30). The layers 346, 348, 350, and 352 are at least substantially maintained in a fixed position relative to each other. The stack 342 may be fabricated to include at least one MEMS filtering section 344, but more typically a plurality of filtering sections 344 as illustrated. FIGS. 3B and 3C illustrate one filtering section 344 that has been “cut out” from the stack 342, while FIGS. 3D-F illustrate the entirety of the various adjacent pairs of adjacent layers within the stack 342 of the MEMS filter module 340.

The stack 342 includes a first layer 346 (e.g., an end layer), a second layer 348 (e.g., an intermediate layer), a third layer 350 (e.g., an intermediate layer), and a fourth layer 352 (e.g., an end layer) progressing from one end of the stack 342 to its opposite end. The layers 346, 348, 350, and 352 are stacked in a first dimension, such that the stack 342 may also be characterized as extending or having its thickness extend in the first dimension as well. The first layer 346 is at one end of the stack 342, and thereby also may be characterized as a first end layer 346. Similarly, the fourth layer 352 is on an opposite end of the stack 342, and thereby also may be characterized as a second end layer 352. The second layer 348 is located or disposed between the first layer 346 and the third layer 350. A space 370 separates the second layer 348 from the third layer 350, while a filter trap 364 separates a portion of the second layer 348 from the first layer 346. The third layer 350 is located or disposed between the second layer 348 and the fourth layer 352. A space 370 separates the third layer 350 from the fourth layer 352.

Portions of the second layer 348 are disposed directly on the first layer 346 (a gap exists between part of the second layer 348 and a corresponding part of the first layer 346 within each filtering section 344, and which defines a filter trap 364). A plurality of structural interconnects 358 extend between and structurally interconnect the third layer 350 and the second layer 348. One or more structural interconnects 358 also extend between and structurally interconnect the fourth layer 352 and the third layer 350. Each individual structural interconnect 358 may be of any appropriate size, shape, and/or configuration. Moreover, the various structural interconnects 358 may be disposed in any appropriate arrangement between the fourth layer 352 and the third layer 350, as well as between the third layer 350 and the second layer 348.

Appropriate seals are provided between the various layers 346, 348, 350, 352. An annular sealing wall 354 extends between and structurally interconnects the second layer 348 and the third layer 350 (FIG. 3E). Another annular sealing wall 354 extends between and structurally interconnects the third layer 350 and the fourth layer 352 (FIG. 3F). The various filtering sections 344 are disposed inwardly of these annular sealing walls 354. The term “annular” herein means that the referenced structure extends a full 360° about a common reference point, and does not limit this structure to having a circular configuration. Various other annular configurations may be appropriate (e.g., square, rectangular, elliptical). An annular sealing wall 354 could also extend between the first layer 346 and the second layer 348 (not shown). However, in the illustrated embodiment a separate filter trap seal 356 between the first layer 346 and the second layer 348 is disposed about each individual filtering section 344. Each filter trap seal 356 is defined by a deposition of the second layer 348 directly onto the first layer 346 in a manner that will be discussed in more detail below.

Each filtering section 344 includes at least one filter trap 364 or at least one filter trap 368 between each adjacent pair of layers 346, 348, 350, 352 within the stack 342. The various filter traps 364, 368 may be of any appropriate size (e.g., to filter a particle having a minimum dimension of at least a certain size), and are preferably of the same size although such may not be required in all instances. The filter traps 364, 368 will each retain at least a substantial portion of objects larger than about 0.4 microns in one embodiment, objects larger than about 0.3 microns in another embodiment, objects larger than about 0.2 microns in another embodiment, and objects larger than about 0.1 microns in yet another embodiment. An annular filter wall 366 extends from the fourth layer 352 in the direction of the third layer 350 in each filtering section 344. A space between this filter wall 366 (the distal end thereof in the illustrated embodiment) and the third layer 350 defines a filter trap 368. An annular filter wall 366 also extends from the third layer 350 in the direction of the second layer 348 in each filtering section 344. A space between this filter wall 366 (the distal end thereof in the illustrated embodiment) and the second layer 348 defines a filter trap 368. Any filter wall 366 could be replaced by a plurality of filter wall segments (not shown) that are appropriately spaced from each other (e.g., corresponding with the size of a filter trap 368).

A filter trap 364 exists between the second layer 348 and the first layer 346 in each filtering section 344, and corresponds with that portion of the second layer 348 that is spaced from the first layer 346. As noted above, a filter trap seal 356 is disposed about the filter trap 364 of each filtering section 344. This filter trap 364 may be defined by: depositing a sacrificial layer (e.g., S₂ layer 20) onto the first layer 346; patterning this sacrificial layer to define the desired shape for the filter trap 364; depositing the second layer 348 directly onto the exposed portions of the first layer 346 and onto the portion of the sacrificial layer that remains on the first layer 346; and removing the remaining portion of the sacrificial layer between the second layer 348 and the first layer 346 to define a filter trap 364. Although the illustrated embodiment uses a single, continuous filter trap 364 for each filtering section 344, such may not be required in all instances (e.g., a plurality of separate filter traps 364 could be provided for each filtering section 344).

Based upon the foregoing, it should be appreciated that each filtering section 344 includes filter traps at three different elevations within the stack 342. Specifically in relation to each filtering section 344, a filter trap 368 that is located between the fourth layer 352 and the third layer 350, which is at a different elevation within the stack 342 than the filter trap 368 that is located between the third layer 352 and the second layer 348, which in turn is at a different elevation within the stack 342 than the filter trap 364 that is located between the second layer 348 and the first layer 346.

At least one flow port extends completely through each of the various layers 346, 348, 350, 352 in each filtering section 344 of the MEMS filter module 340. Each such flow port is either characterized herein as a Group I flow port 360 or a Group II flow port 362. Both the Group I flow ports 360 and Group II flow ports 362 may be of any appropriate size, shape, and/or configuration, and may be disposed in any appropriate arrangement. In the illustrated embodiment, each Group II flow port 362 is the same size, although such may not be required in all instances. In any case, each flow port 360, 362 preferably provides less flow resistance than any corresponding filter trap 364, 368 (e.g., each flow port 360, 362 is “larger” than any corresponding filter trap 364, 368 in a dimension that is orthogonal to the flow therethrough).

A flow through any Group I flow port 360 may proceed to another Group I flow port 360 in any adjacent layer without passing through either a filter trap 364 or a filter trap 368. Similarly, a flow through any Group II flow port 362 may proceed to another Group II flow port 362 in any adjacent layer without passing through either a filter trap 364 or a filter trap 368. However, in order for a flow to proceed from a Group I flow port 360 to any Group II flow port 362 in an adjacent layer or vice versa, this flow must proceed through either a filter trap 364 or a filter trap 368 in the case of the MEMS filter module 340.

The flow ports 360, 362 associated with the MEMS filter module 340 are arranged to accommodate a desirably high flow rate through the MEMS filter module 340, and yet still provide a suitable filtering function. Generally, one reference layer of the MEMS filter module 340 (an end layer in the illustrated embodiment, although it is possible that this reference layer may not define an end of the MEMS filter module 340) will contain only Group I flow ports 360, another reference layer of the MEMS filter module 340 (an end layer in the illustrated embodiment, although it is possible that this reference layer may not define an end of the MEMS filter module 340) will have only Group II flow ports 362, and each intermediate layer (those located between the two reference layers) will have both Group I flow ports 360 and Group II flow ports 362. In the illustrated embodiment, each filtering section 344 of the MEMS filter module 340 includes the following in relation to the flow ports 360, 362: the first layer 346 includes at least one Group I flow port 360 (one in the illustrated embodiment), but does not include any Group II flow ports 362; the second layer 348 and the third layer 350 each include at least one Group I flow port 360 (one in the illustrated embodiment) and at least one Group II flow port 362 (a plurality in the illustrated embodiment); and the fourth layer 352 includes at least one Group II flow port 362 (a plurality in the illustrated embodiment), but does not include any Group I flow ports 360.

A number of additional observations may be made in relation to the flow ports 360, 362 for each filtering section 344 in the illustrated embodiment: each Group I flow port 360 is axially aligned with a Group I flow port 360 in each adjacent layer, although such may not be required in all instances; the size of the Group I flow ports 360 progressively changes proceeding through the stack 342, although such may not be required in all instances (the Group I flow ports 360 get progressively smaller proceeding in the direction of the first layer 346 from the fourth layer 352 in the illustrated embodiment); and each Group II flow port 362 is axially aligned with a Group II flow port 362 in each adjacent layer, although such may not be required in all instances.

The MEMS flow module 340 will accommodate a bidirectional flow. With further regard to the flow through the MEMS filter module 340, the flow through the various flow ports 360, 362 is at least generally within the first dimension (the “thickness” dimension of the stack 342), while the flow through each filter trap 364, 368 is at least generally within a second dimension that is different than the first dimension. In the illustrated embodiment, the flow through each filter trap 364, 368 is at least generally orthogonal to the flow through the flow ports 360,362.

A desirably high flow rate may proceed through the MEMS filter module 340 and yet still be appropriately filtered as noted. Consider the case of a flow through one of the Group II flow ports 362 of the fourth layer 352 in a particular filtering section 344. This flow may proceed through filter traps 364, 368 at three different elevations and reach a Group I flow port 360 of the first layer 346 in order to exit the MEMS filter module 340. For instance, this flow could proceed through a filter trap 368 between the fourth layer 352 and the third layer 350, and then through a Group I flow port 360 in each of the third layer 350, the second layer 348, and the first layer 346. Another option would be for this flow to proceed through a Group II flow port 362 of the third layer 350 (which provides less flow resistance than a filter trap 368 between the fourth layer 352 and the third layer 350), then through the filter trap 368 between the third layer 350 and the second layer 348, and then through a Group I flow port 360 in each of the second layer 348 and the first layer 346. Yet another option would be for this flow to proceed through a Group II flow port 362 of the third layer 350 (which provides less flow resistance than a filter trap 364 between the fourth layer 352 and the third layer 350), then through a Group II flow port 362 of the second layer 348 (which provides less flow resistance than a filter trap 368 between the third layer 350 and the second layer 348), then through a filter trap 364 between the second layer 348 and the first layer 346, and then through a Group I flow port 360 in the first layer 346. It should also be appreciated that a flow through any Group II flow port 362 of the fourth layer 352 and through any Group II flow port 362 of the third layer 350 from one filtering section 344 may in fact proceed to other filtering sections 344 in the illustrated embodiment. The MEMS filter module 340 could be configured such that a flow through any Group II flow port 362 of the second layer 352 from one filtering section 344 could proceed to other filtering sections 344 as well, although this is not the case in the illustrated embodiment.

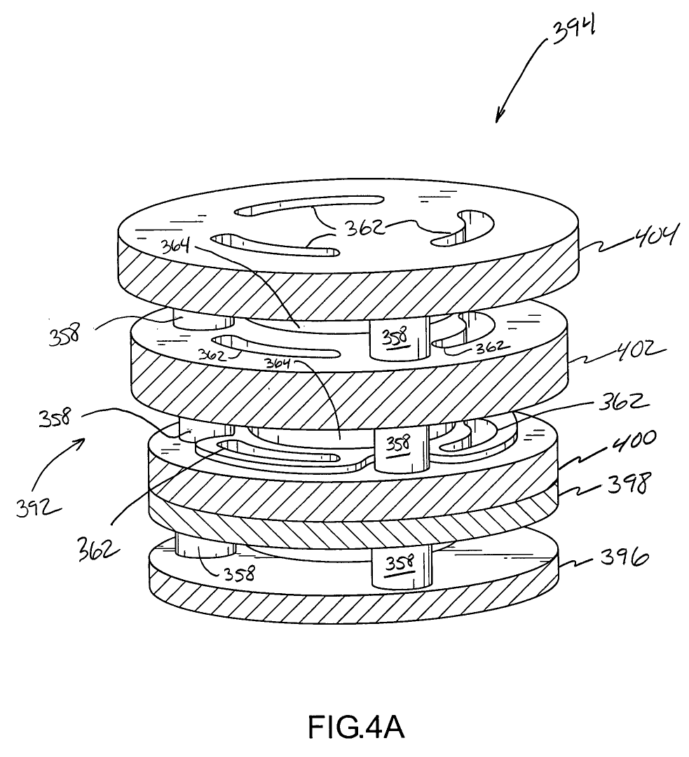

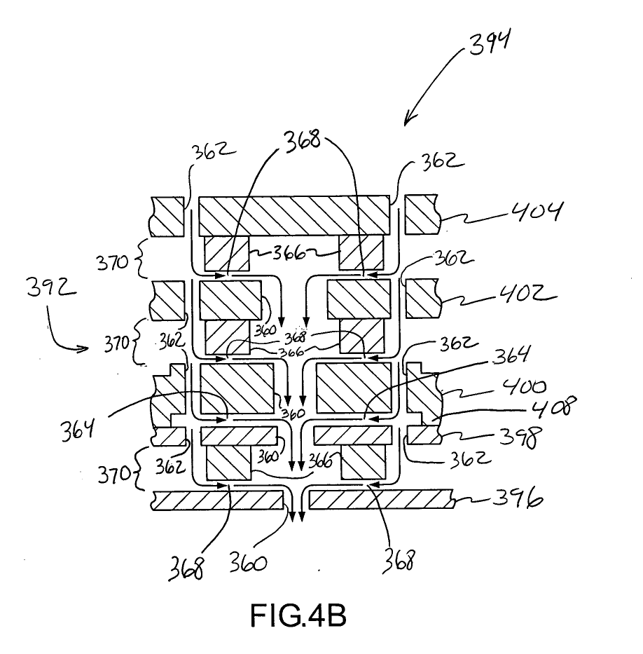

Another embodiment of a MEMS filtering section that includes a plurality of filter traps at each of a plurality of levels is illustrated in FIGS. 4A-B, is identified by reference numeral 390, and in effect is a variation of the MEMS filtering section 344 used by the MEMS filter module 340 discussed above in relation to FIGS. 3A-F. The MEMS filtering section 390 of FIGS. 4A-B could be used in place of the MEMS filtering section 344 for the MEMS filter module 340 or any other MEMS flow module.

The primary difference between the filtering section 344 from the MEMS filter module 340 of FIGS. 3A-F and the filtering section 394 of FIGS. 4A-B is the addition of filter traps at an additional level or elevation (4 different levels for the filtering section 394 of FIGS. 4A-B versus 3 different levels for the filtering section 344 of FIGS. 3A-F). At least certain common components between these embodiments are identified by the same reference numeral, and the discussion presented above with regard to these components for the MEMS flow module 340 will remain equally applicable to a MEMS flow module that uses the filtering section 394 unless otherwise noted. The filtering section 394 is in the form of a stack 392 of a plurality of layers 396 (e.g., dielectric layer 12—P₀ layer 14 may be disposed on dielectic layer 12 as well), 398 (e.g., P₁ layer 18), 400 (e.g., P₂ layer 22), 402 (e.g., P₃ layer 26), and 404 (e.g., P₄ layer 30). The layers 396, 398, 400, 402, and 404 are at least substantially maintained in a fixed position relative to each other. A MEMS flow module could include a single filtering section 394, but more typically would include a plurality of filtering sections 394.

The stack 392 includes a first layer 396 (e.g., an end layer), a second layer 398 (e.g., an intermediate layer), a third layer 400 (e.g., an intermediate layer), a fourth layer 402 (e.g., an intermediate layer), and an fifth layer 404 (e.g., an end layer) progressing from one end of the stack 392 to its opposite end. The layers 396, 398, 400, 402, and 404 are stacked in a first dimension, such that the stack 392 may also be characterized as extending or having its thickness extend in the first dimension as well. The first layer 396 is at one end of the stack 392, and thereby also may be characterized as a first end layer 396. Similarly, the fifth layer 404 is on an opposite end of the stack 392, and thereby also may be characterized as a second end layer 404. The second layer 398 is located or disposed between the first layer 396 and the third layer 400. A filter trap 364 separates a portion of the third layer 400 from the second layer 346, while a space 370 separates the second layer 398 from the first layer 396. The third layer 400 is located or disposed between the second layer 398 and the fourth layer 402. A space 370 separates the third layer 400 from the fourth layer 402. The fourth layer 402 is located or disposed between the third layer 400 and the fifth layer 404. A space 370 separates the fourth layer 402 from the fifth layer 404.

Portions of the third layer 400 are disposed directly on the second layer 398 (a gap exists between part of the third layer 400 and a corresponding part of the second layer 398 within each filtering section 394, and which defines a filter trap 364). One or more structural interconnects 358 extend between and structurally interconnect the first layer 396 and the second layer 398. One or more structural interconnects 358 extend between and structurally interconnect the third layer 400 and the fourth layer 402. One or more structural interconnects 358 also extend between and structurally interconnect the fourth layer 402 and the fifth layer 404. Each individual structural interconnect 358 may be of any appropriate size, shape, and/or configuration. Moreover, the various structural interconnects 358 may be disposed in any appropriate arrangement between the fifth layer 404 and the fourth layer 402, between the fourth layer 402 and the third layer 400, as well as between the second layer 398 and the first layer 396.

Appropriate seals would be provided between the various layers 396, 398, 400, 402, and 404 for a MEMS flow module that includes at least one filtering section 394 and in accordance with the MEMS filter module 340 discussed above. An annular sealing wall 354 (not shown) could extend between and structurally interconnect the first layer 396 and the second layer 398. Another annular sealing wall 354 (not shown) could extend between and structurally interconnect the third layer 400 and the fourth layer 402. Yet another annular sealing wall 354 (not shown) could extend between and structurally interconnect the fourth layer 402 and the fifth layer 404. Each filtering section 394 used by a MEMS flow module would be disposed inwardly of these annular sealing walls 354. An annular sealing wall 354 could also extend between the third layer 396 and the second layer 398 (not shown). However, in the illustrated embodiment a separate filter trap seal 408 is disposed about each filtering section 394. Each filter trap seal 408 is defined by a deposition of the third layer 400 directly onto the second layer 398 in the same general manner discussed above with regard to the second layer 348 and first layer 346 in the case of the MEMS flow module 340.

Each filtering section 394 includes at least one filter trap 364 or at least one filter trap 368 between each adjacent pair of layers 396, 398, 400, 402, and 404 within the stack 392. An annular filter wall 366 extends from the fifth layer 404 in the direction of the fourth layer 402 in each filtering section 394. A space between this filter wall 366 (the distal end thereof in the illustrated embodiment) and the fourth layer 402 defines a filter trap 368. An annular filter wall 366 extends from the fourth layer 402 in the direction of the third layer 400 in each filtering section 394. A space between this filter wall 366 (the distal end thereof in the illustrated embodiment) and the third layer 400 defines a filter trap 368. An annular filter wall 366 also extends from the second layer 398 in the direction of the first layer 396 in each filtering section 394. A space between this filter wall 366 (the distal end thereof in the illustrated embodiment) and the first layer 396 defines a filter trap 368.

A filter trap 364 exists between the third layer 400 and the second layer 398 in each filtering section 394, and corresponds with that portion of the third layer 400 that is spaced from the second layer 398. As noted above, a filter trap seal 408 is disposed about the filter trap 364 of each filtering section 394, although such need not always be the case. This filter trap 364 may be defined by: depositing a sacrificial layer (e.g., S₂ layer 20) onto the second layer 398 (e.g., P₁ layer 18); patterning this sacrificial layer to define the desired shape for the filter trap 364; depositing the third layer 400 (e.g., P₂ layer 22) directly onto the exposed portions of the first layer 396 and onto the portion of the sacrificial layer that remains on the first layer 396; and removing the remaining portion of the sacrificial layer between the third layer 400 and the second layer 398 to define a filter trap 364. Although the illustrated embodiment uses a single, continuous filter trap 364 for each filtering section 394, such may not be required in all instances (e.g., a plurality of separate filter traps 364 could be provided for each filtering section 394).

Based upon the foregoing, it should be appreciated that each filtering section 394 includes filter traps at four different elevations within the stack 392. That is, the filter trap 368 between the fifth layer 404 in the fourth layer 402 is at a different elevation than the filter trap 368 that is between the fourth layer 402 and the third layer 400, which in turn is at a different elevation than the filter trap 364 between the third layer 400 and the second layer 398, which in turn is at a different elevation than the filter trap 368 between the second layer 398 and the first layer 396.

At least one Group I flow port 360 or Group II flow port 362 extends completely through each of the various layers 396, 398, 400, 402, 404 in the filtering section 394. The flow ports 360, 362 associated with the filtering section 394 are arranged to accommodate a desirably high flow rate through a MEMS flow module using one or more filtering sections 394, and yet still provide a suitable filtering function. As in the case of the MEMS filter module 340 of FIGS. 3A-F, one reference layer of the filtering section 394 (an end layer in the illustrated embodiment, although it is possible that this reference layer may not define an end of a MEMS flow module that uses one or more filtering sections 394) will contain only Group I flow ports 360, another reference layer of the filtering section 394 (an end layer in the illustrated embodiment, although it is possible that this reference layer may not define an end of a MEMS flow module that uses one or more filtering sections 394) will have only Group II flow ports 362, and each intermediate layer (those located between the two reference layers) will have both Group I flow ports 360 and Group II flow ports 362. In the illustrated embodiment, each filtering section 394 includes the following in relation to the flow ports 360, 362: the first layer 396 includes at least one Group I flow port 360 (one in the illustrated embodiment), but does not include any Group II flow ports 362; the second layer 398, the third layer 400, and the fourth layer 402 each include at least one Group I flow port 360 (one in the illustrated embodiment) and at least one Group II flow port 362 (a plurality in the illustrated embodiment); and the fifth layer 404 includes at least one Group II flow port 362 (a plurality in the illustrated embodiment), but does not include any Group I flow ports 360.

A MEMS flow module that utilizes one or more filtering sections 394 will accommodate a bidirectional flow. A desirably high flow rate may proceed through a filtering section 394 and yet still be appropriately filtered as noted. Consider the case of a flow through one of the Group II flow ports 362 of the fifth layer 404 in a particular filtering section 394. This flow may proceed through filter traps 364, 368 at four different elevations and reach a Group I flow port 360 of the first layer 396 in order to exit the filtering section 394. For instance, this flow could proceed through a filter trap 368 between the fifth layer 404 and the fourth layer 402, and then through a Group I flow port 360 in each of the fourth layer 402, the third layer 400, the second layer 398, and the first layer 396. Another option would be for this flow to proceed through a Group II flow port 362 of the fourth layer 402 (which provides less flow resistance than a filter trap 368 between the fifth layer 404 and the fourth layer 402), then through the filter trap 368 between the fourth layer 402 and the third layer 400, and then through a Group I flow port 360 in each of the third layer 400, the second layer 398, and the first layer 396. Another option would be for this flow to proceed through a Group II flow port 362 of the fourth layer 402 (which provides less flow resistance than a filter trap 368 between the fifth layer 404 and the fourth layer 402), then through a Group II flow port 362 of the third layer 400 (which provides less flow resistance than a filter trap 368 between the fourth layer 402 and the third layer 400), then through the filter trap 364 between the third layer 400 and the second layer 398, and then through a Group I flow port 360 in each of the second layer 398 and the first layer 396. Yet another option would be for this flow to proceed through a Group II flow port 362 of the fourth layer 402 (which provides less flow resistance than a filter trap 368 between the fifth layer 404 and the fourth layer 402), through a Group II flow port 362 of the third layer 400 (which provides less flow resistance than a filter trap 368 between the fourth layer 402 and the third layer 400), then through a Group II flow port 362 of the second layer 398 (which provides less flow resistance than a filter trap 364 between the third layer 400 and the second layer 398), then through a filter trap 368 between the second layer 398 and the first layer 396, and then through a Group I flow port 360 of the first layer 396. It should also be appreciated that a flow through any Group II flow port 362 of the fifth layer 404, of the fourth layer 402, and of the second layer 398 from one filtering section 394 may in fact proceed to other filtering sections 394 of a MEMS flow module that utilizes a plurality of these filtering sections 394. The filtering section 394 could be configured such that a flow through any Group II flow port 362 of the third layer 400 from one filtering section 394 could proceed to other filtering sections 394 in the same MEMS flow module, although this is not the case in the illustrated embodiment.

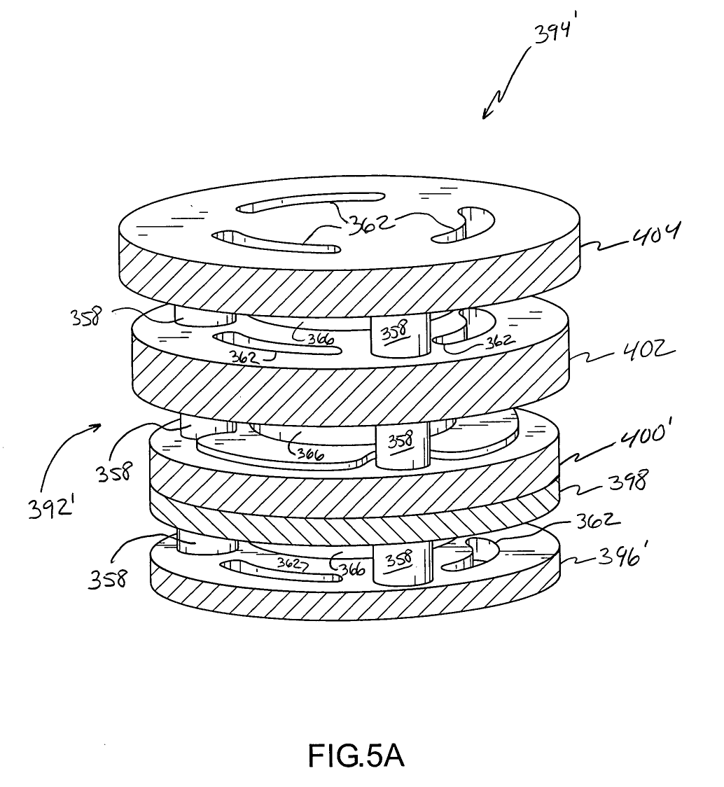

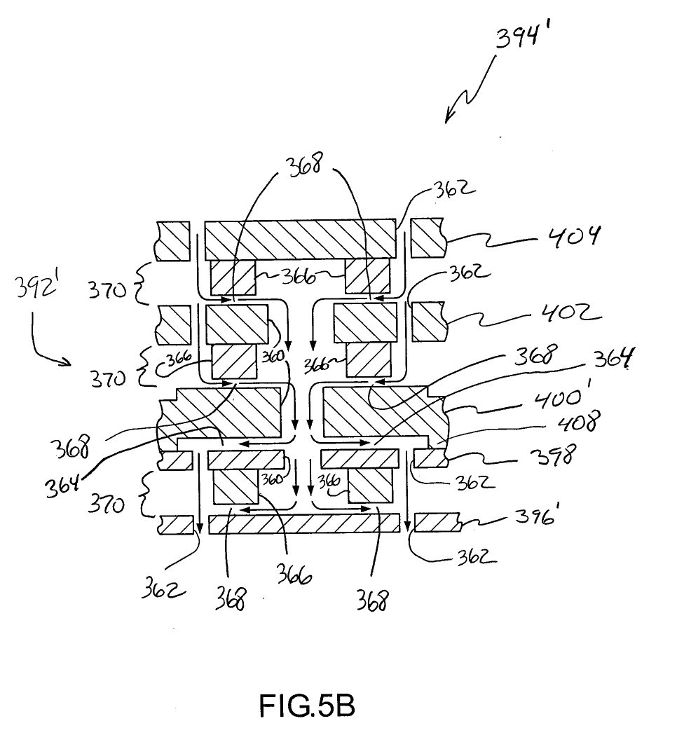

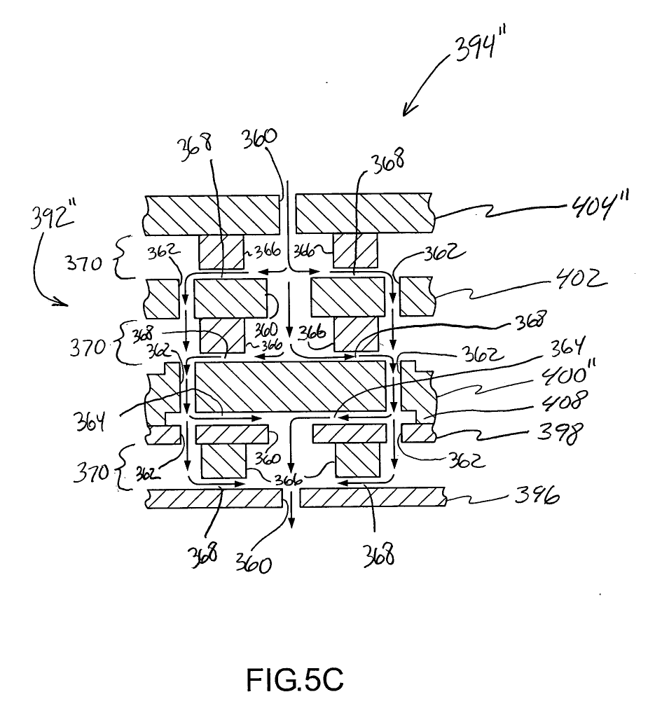

Another embodiment of a MEMS filtering section that includes a plurality of filter traps at each of a plurality of levels is illustrated in FIGS. 5A-B, is identified by reference numeral 394′, and is a variation of the filtering section 394 that was discussed above in relation to FIGS. 4A-B. Corresponding components between these two embodiments are identified by the same 5 reference numeral, and the discussion presented above with regard to these components will remain equally applicable unless otherwise noted. Those corresponding components that differ in at least some respect are identified with the same reference numeral, along with a single prime designation. The filtering section 394 of FIGS. 5A-B could be used in place of the filtering section 344 for the MEMS filter module 340 or any other MEMS flow module.

Each filtering section 394′ in the case of the embodiment of FIGS. 5A-B still includes filter traps at four different elevations within the stack 392′ of layers 404 (e.g., an end layer), 402 (e.g., an intermediate layer), 400′ (e.g., a “first layer”; an intermediate layer), 398 (e.g., an intermediate layer; a first sub layer), and 396′ (e.g., an end layer; a second sub layer). That is, the filter trap 368 between the fifth layer 404 and the fourth layer 402 is at a different elevation than the filter trap 368 that is between the fourth layer 402 and the third layer 400′, which in turn is at a different elevation than the filter trap 364 between the third layer 400′ and the second layer 398, which in turn is at a different elevation than the filter trap 368 between the second layer 398 and the first layer 396′. The filtering section 394′ of FIGS. 5A-B will also accommodate a bidirectional flow. The manner in which a flow may progress through the filtering section 394′of FIGS. 5A-B is different than for the case of the filtering section 394 of FIGS. 4A-B.

The primary difference between the filtering 394 of FIGS. 4A-B and the filtering section 394′ of FIGS. 5A-B is all flow through the filtering section 394′ is required to pass through a filter trap at each of two different elevations in order to progress through the filtering section 394′. This is accomplished by modifying the first layer 396′ and the third layer 400′ of the filtering section 394′. The first layer 396′ of the filtering section 394′ includes at least one Group II flow port 362 (a plurality in the illustrated embodiment), but does not include any Group I flow ports 360. The third layer 400′ of the filtering section 394′ includes at least one Group I flow port 360 (one in the illustrated embodiment), but does not include any Group II flow ports 362.

A desirably high flow rate may proceed through the filtering section 394′, and the potential for undesired particulates proceeding therethrough is reduced by requiring all flow to pass through filter traps 364, 368 at each of two different elevations in order to progress completely through the filtering section 394′. Consider the case of a flow through one of the Group II flow ports 362 of the fifth layer 404 in a particular filtering section 394′. This flow may proceed through any number of the filter traps 368 and reach a Group I flow port 360 of the third layer 400′ (an intermediate point in the progression through the filtering section 394′). For instance, this flow could proceed through a filter trap 368 between the fifth layer 404 and the fourth layer 402, and then through a Group I flow port 360 in the fourth layer 402 to reach a Group I flow port 360 of the third layer 400. Another option would be for this flow to proceed through a Group II flow port 362 of the fourth layer 402 (which provides less flow resistance than a filter trap 368 between the fifth layer 404 and the fourth layer 402), and then through the filter trap 368 between the fourth layer 402 and the third layer 400′ to reach a Group I flow port 360 of the third layer 400.

A flow through any Group I flow port 360 of the third layer 400′ may proceed through filter traps 364, 368 at two different elevations and reach a Group II flow port 362 of the first layer 396 to exit the filtering section 394′. For instance, this flow could proceed through a filter trap 364 between the third layer 400′ and the second layer 398, and then through a Group II flow port 362 in each of the second layer 398 and the first layer 396′. Another option would be for this flow to proceed through a Group I flow port 360 of the second layer 398 (which provides less flow resistance than a filter trap 364 between the third layer 400′ and the second layer 398), and then through the filter trap 368 between the second layer 398 and the first layer 396′ to reach a Group II flow port 362 of the first layer 396′.

Another embodiment of a MEMS filtering section that includes a plurality of filter traps at each of a plurality of levels is illustrated in FIG. 5C, is identified by reference numeral 394″, and is a variation of the filtering section 394′ that was discussed above in relation to FIGS. 5A-B discussed above, which in turn is a variation of the filtering section 344 that was discussed above in relation to FIGS. 4A-B. Corresponding components between these embodiments are identified by the same reference numeral, and the discussion presented above with regard to these components will remain equally applicable unless otherwise noted. Those corresponding components of the filtering section 394″ that differ in at least some respect from the filtering section 344 of FIGS. 4A-B are identified with the same reference numeral, along with a double prime designation. The filtering section 390″ of FIG. 5C could be used in place of the filtering section 344 for the MEMS filter 340 or any other MEMS flow module

The filtering section 394″ still includes filter traps at four different elevations within the stack 392″ of layers 404″ (e.g., an end layer; a “first layer”), 402 (e.g., an intermediate layer), 400″ (e.g., a “third layer”; an intermediate layer), 398 (e.g., an intermediate layer; a first sub layer), and 396 (e.g., an end layer; a second sub layer). That is, the filter trap 368 between the fifth layer 404″ and the fourth layer 402 is at a different elevation than the filter trap 368 that is between the fourth layer 402 and the third layer 400″, which in turn is at a different elevation than the filter trap 364 between the third layer 400″ and the second layer 398, which in turn is at a different elevation than the filter trap 368 between the second layer 398 and the first layer 396. The filtering section 394″ will also accommodate a bidirectional flow. The manner in which a flow may progress through the filtering section 394″ of FIG. 5C is different than for the case of the filtering section 344 of FIGS. 4A-B.

The primary difference between the filtering section 344 of FIGS. 4A-B and the filtering section 394″ of FIG. 5C is all flow through the filtering section 394″ is required to pass through a filter trap at each of two different elevations in order to progress through the filtering section 394″. This is accomplished by modifying the third layer 400″ and the fifth layer 404″ of the filtering section 394″. The fifth layer 404″ of the filtering section 394″ includes at least one Group I flow port 360, but does not include any Group II flow ports 362. The third layer 400″ of the filtering section 394″ includes at least one Group II flow port 362 (a plurality in the illustrated embodiment), but does not include any Group I flow ports 360.

A desirably high flow rate may proceed through the filtering section 394″, and the potential for undesired particulates proceeding therethrough is reduced by requiring all flow to pass through filter traps 364, 368 at each of two different elevations in order to progress completely through the filtering section 394″. Consider the case of a flow through one of the Group I flow ports 360 of the fifth layer 404″ in a particular filtering section 394″. This flow may proceed through any number of the filter traps 368 and reach a Group II flow port 362 of the third layer 400″ (an intermediate point in the progression through the filtering section 394″). For instance, this flow could proceed through a filter trap 368 between the fifth layer 404″ and the fourth layer 402, and then through a Group II flow port 362 in the fourth layer 402 to reach a Group II flow port 362 of the third layer 400″. Another option would be for this flow to proceed through a Group I flow port 360 of the fourth layer 402 (which provides less flow resistance than a filter trap 368 between the fifth layer 404″ and the fourth layer 402), and then through the filter trap 368 between the fourth layer 402 and the third layer 400″ to reach a Group II flow port 362 of the third layer 400″.

A flow through any Group II flow port 362 of the third layer 400″ may proceed through filter traps 364, 368 at two different elevations and reach a Group I flow port 360 of the first layer 396 to exit the filtering section 394″. For instance, this flow could proceed through a filter trap 364 between the third layer 400″ and the second layer 398, and then through a Group I flow port 360 in each of the second layer 398 and the first layer 396. Another option would be for this flow to proceed through a Group II flow port 362 of the second layer 398 (which provides less flow resistance than a filter trap 364 between the third layer 400″ and the second layer 398), and then through the filter trap 368 between the second layer 398 and the first layer 396 to reach a Group I flow port 360 of the first layer 396.

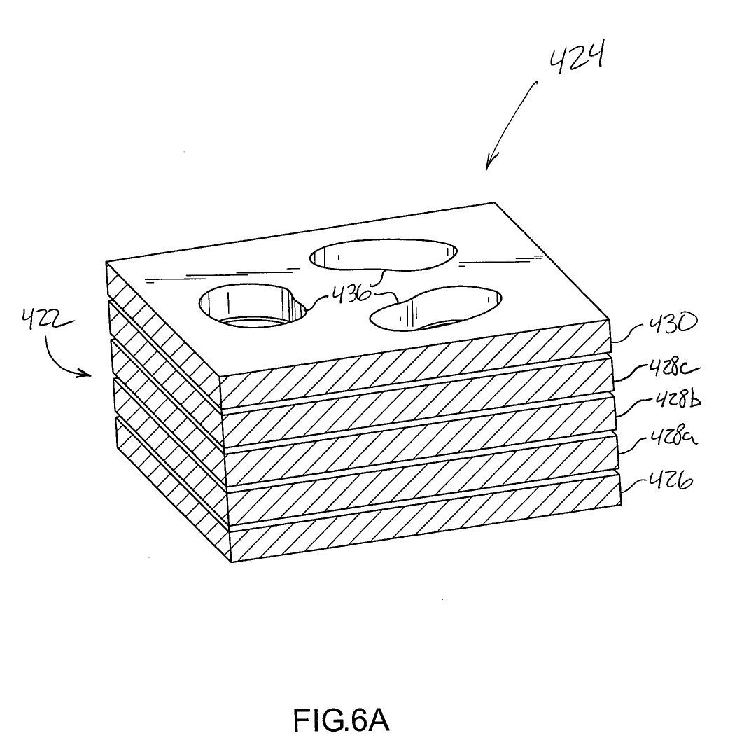

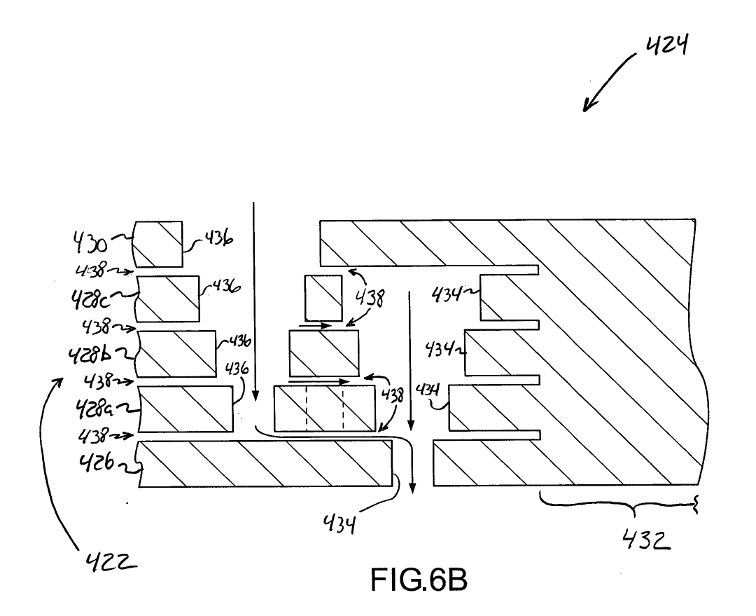

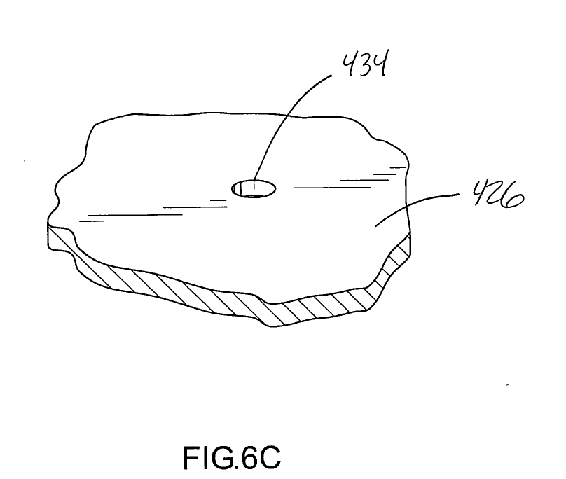

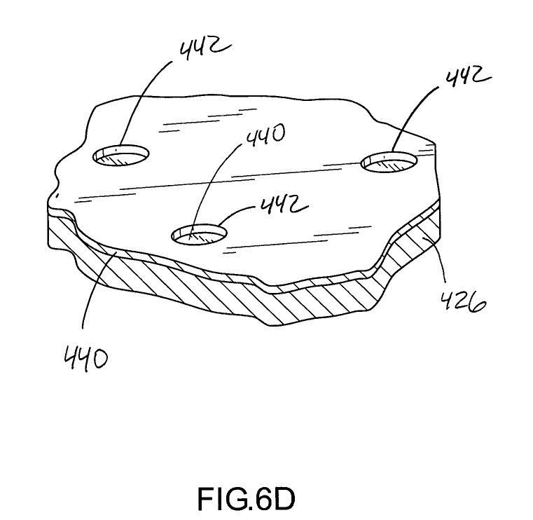

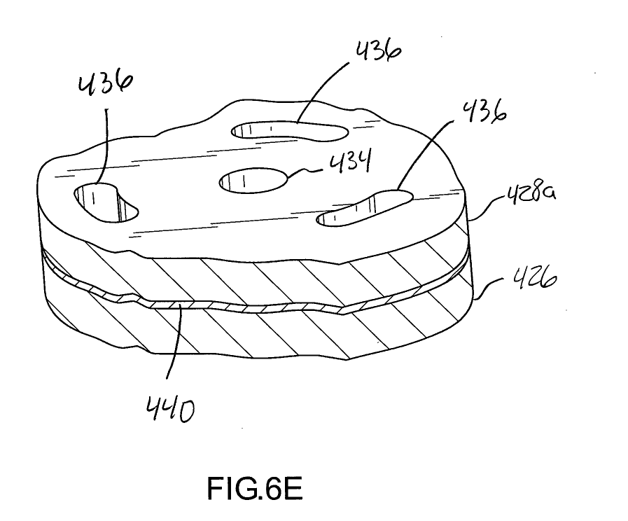

Another embodiment of a MEMS filtering section that includes a plurality of filter traps at each of a plurality of levels is illustrated in FIGS. 6A-B and is identified by reference numeral 424. The filtering section 424 of FIGS. 6A-B could be used in place of the filtering section 344 for the MEMS filter module 340 or any other MEMS flow module. The MEMS filtering section 424 is in the form of a stack 422 of layers 426, 428a-c, and 430. The layers 426, 428a-c, and 430 are at least substantially maintained in a fixed position relative to each other. The stack 422 may be fabricated to include a single filtering section 424, but more typically will include a plurality of filtering sections 424.

The layers 426, 428a-c, and 430 are stacked in a first dimension, such that the stack 422 may also be characterized as extending or having its thickness extend in the first dimension as well. The layer 426 is at one end of the stack 422, and thereby also may be characterized as a first end layer 426. Similarly, the layer 430 is on an opposite end of the stack 422, and thereby also may be characterized as a second end layer 430. The layers 428a-c are located or disposed between the first end layer 426 and the second end layer 430, and thereby these may be referred to as intermediate layers 428a-c. Any appropriate number of intermediate layers 428a-c could be utilized.

The various layers 426, 428a-c, and 430 are disposed in spaced relation to each other. More specifically, a filter trap 438 is disposed between adjacent pair of the layers 426, 428a-c, and 430 in the filtering section 424. One or more structural interconnects 432 extend between and structurally interconnect each adjacent pair of layers 426, 428a-c, and 430 in the filtering section 424 as well. Each individual structural interconnect 432 may be of any appropriate size, shape, and/or configuration. Moreover, the various structural interconnects 432 may be disposed in any appropriate arrangement between the various layers 426, 428a-c, and 430 of the filtering section 424. In the illustrated embodiment, the structural interconnects 432 between the various layers 426, 428a-c, and 430 are aligned.

Appropriate seals would typically be provided between each adjacent pair of the various layers 426, 428a-c, and 430 for a MEMS flow module having one or more of the filtering sections 424. An annular sealing wall (not shown, but in accordance with annular sealing wall 354 noted above) could extend between and structurally interconnect each adjacent pair of layers 426, 428a-c, and 430. Each filtering section 424 used by the MEMS flow module would be disposed inwardly of these annular sealing walls. A separate annular sealing wall could also be disposed about each filtering section 424 to fluidly isolate the filtering sections 424 from each other in the case of a MEMS flow module that uses a plurality of filtering sections 424.

Each filtering section 424 includes a filter trap 438 between each adjacent pair of the layers 426, 428a-c, and 430 within the filtering section 424. The various filter traps 438 may be of any appropriate height (corresponding with the magnitude of the gap between an adjacent pair of the layers 426, 428a-c, and 430 within the stack 422), and are preferably of the same height although such may not be required in all instances. The length of the filter traps 438 changes from layer-to-layer in the illustrated embodiment, although such may not be required in all instances. The filter traps 438 will each retain at least a substantial portion of objects larger than about 0.4 microns in one embodiment, objects larger than about 0.3 microns in another embodiment, objects larger than about 0.2 microns in another embodiment, and objects larger than about 0.1 microns in yet another embodiment.

The filtering section 424 includes filter traps 438 at four different elevations. That is, the filter trap 438 between the second end layer 430 and the intermediate layer 428c is at a different elevation than the filter trap 438 that is between the intermediate layer 428c and the intermediate layer 428b, which in turn is at a different elevation than the filter trap 438 that is between the intermediate layer 428b and the intermediate layer 428a, which in turn is at a different elevation than the filter trap 438 that is between the intermediate layer 428a and the first end layer 426.

The size of each filter trap 438 is defined by the spacing between the adjacent pair of layers that define the filter trap 438. For instance, the spacing between the first end layer 426 and the intermediate layer 428a defines the size of one filter trap 438, while the spacing between the intermediate layer 428a and the intermediate layer 428b defines the size of another filter trap s 438 that is disposed at a different elevation within the stack 422. Preferably: a constant spacing (of a common dimension) exists between the entirety of the first end layer 426 and the intermediate layer 428a that are disposed in spaced relation such that the entirety of the corresponding filter trap 438 defined therebetween is of a constant size as well; a constant spacing (of a common magnitude) exists between the entirety of the intermediate layer 428a and the intermediate layer 428b that are disposed in spaced relation such that the entirety of the corresponding filter trap 438 defined therebetween is of a constant size as well; a constant spacing (of a common magnitude) exists between the intermediate layer 428b and the intermediate layer 428c that are disposed in spaced relation such that the entirety of the corresponding filter trap 438 defined therebetween is of a constant size as well; a constant spacing (of a common magnitude) exists between the entirety of the intermediate layer 428c and the second end layer 430 that are disposed in spaced relation such that the entirety of the corresponding filter trap 438 defined therebetween is of a constant size as well. Stated another way, the stack 422 includes a plurality of at least substantially planar surfaces (those surfaces of each of the layers 426, 428a-c, and 430 that face another layer 426, 428a-c, and 430 within the stack 422), that are disposed in at least substantially parallel relation, and that define the various filter traps 438. Yet another characterization regarding each filter trap 438 is that it is defined by the maximum spacing between an adjacent pair of layers in the stack 422.

At least one flow port extends completely through each of the various layers 426, 428a-c, and 430 in each filtering section 424 of the stack 422. Each such flow port is either characterized herein as a Group I flow port 434 or a Group II flow port 436. Each flow port 434, 436 provides less flow resistance than its associated filter traps 438 (e.g., each flow port 434, 436 is “larger” than each corresponding filter trap 438 in a dimension that is orthogonal to the flow therethrough). Both the Group I flow ports 434 and Group II flow ports 436 may be of any appropriate size, shape, and/or configuration. A flow through any Group I flow port 434 may proceed to another Group I flow port 434 in any adjacent layer without passing through a filter trap 438. Similarly, a flow through any Group II flow port 436 may proceed to another Group II flow port 436 in any adjacent layer without passing through a filter trap 438. However, in order for a flow to proceed from a Group I flow port 434 to any Group II flow port 436 in an adjacent layer or vice versa, this flow must proceed through a filter trap 438 in the case of the filtering section 424.

The flow ports 434, 438 associated with the filtering section 424 are arranged to accommodate a desirably high flow rate through the filtering section 424, and yet still provide a suitable filtering function. In the illustrated embodiment, each filtering section 424 in the stack 422 includes the following in relation to the flow ports 434, 436: the first end layer 426 includes at least one Group I flow port 434 (one in the illustrated embodiment), but does not include any Group II flow ports 436; each intermediate layer 428a-c includes at least one Group I flow port 434 (one in the illustrated embodiment) and at least one Group II flow port 436 (a plurality in the illustrated embodiment); and the second end layer 430 includes at least one Group II flow port 436 (a plurality in the illustrated embodiment), but does not include any Group I flow ports 434.

A number of additional observations may be made in relation to the flow ports 434, 436 for each filtering section 424in the illustrated embodiment: each Group I flow port 424 is axially aligned with a Group I flow port 424 in each adjacent layer, although such may not be required in all instances; the size of the Group I flow ports 434 progressively changes proceeding through the stack 422, although such may not be required in all instances (the Group I flow ports 434 get progressively smaller proceeding in the direction of the first end layer 426 from the second end layer 430 in the illustrated embodiment); each Group II flow port 436 is axially aligned with a Group II flow port 436 in each adjacent layer, although such may not be required in all instances; and the size of the Group II flow ports 436 progressively changes proceeding through the stack 422, although such may not be required in all instances (the Group II flow ports 436 get progressively smaller proceeding in the direction of the first end layer 426 from the second end layer 430 in the illustrated embodiment).

Based upon the above-noted progressive change in size for both the Group I flow ports and Group II flow ports, the length of the filter traps 438 also progressively changes from layer-to-layer proceeding through the stack 422. Generally, the filter traps 438 get progressively longer proceeding in the direction of the first end layer 426 from the second end layer 430 in the illustrated embodiment. In order to reduce the resistance of a particular filter trap 438, it may be desirable to fabricate or otherwise provide a cavity at an intermediate location along the length of such a filter trap (e.g., create a recess at a location between the dashed lines in FIG. 6B).

The filtering section 424 will accommodate a bidirectional flow. With further regard to the flow through the filtering section 424, the flow through the various flow ports 434, 436 is at least generally within the first dimension (the “thickness” dimension of the stack 422), while the flow through each filter trap 438 is at least generally within a second dimension that is different than the first dimension. In the illustrated embodiment, the flow through each filter trap 438 is at least generally orthogonal to the flow through the flow ports 434, 436.

A desirably high flow rate may proceed through the filtering section 424 and yet still be appropriately filtered as noted. Consider the case of a flow through one of the Group II flow ports 436 of the second end layer 430 in a particular filtering section 424. This flow may proceed through filter traps 438 at four different elevations and reach a Group I flow port 434 of the first end layer 426 in order to exit the filtering section 424. For instance, this flow could proceed through a filter trap 438 between the second end layer 430 and the intermediate layer 428c, and then through a Group I flow port 434 in each of the intermediate layer 428c, the intermediate layer 428b, the intermediate layer 428a, and the first end layer 426. Another option would be for this flow to proceed through a Group II flow port 436 of the intermediate layer 428c (which provides less flow resistance than a filter trap 438 between the second end layer 430 and the intermediate layer 428c), then through the filter trap 438 between the intermediate layer 428c and the intermediate layer 428b, and then through a Group I flow port 434 in each of the intermediate layer 428b, the intermediate layer 428a, and the first end layer 426. Another option would be for this flow to proceed through a Group II flow port 436 of the intermediate layer 428c (which provides less flow resistance than a filter trap 438 between the second end layer 430 and the intermediate layer 428c), then through a Group II flow port 436 of the intermediate layer 428b (which provides less flow resistance than a filter trap 438 between the intermediate layer 428c and the intermediate layer 428b), then through the filter trap 438 between the intermediate layer 428b and the intermediate layer 428a, and then through a Group I flow port 434 in each of the intermediate layer 428a and the first end layer 426. Yet another option would be for this flow to proceed through a Group II flow port 436 of the intermediate layer 428c (which provides less flow resistance than a filter trap 438 between the second end layer 430 and the intermediate layer 428c), then through a Group II flow port 436 of the intermediate layer 428b (which provides less flow resistance than a filter trap 438 between the intermediate layer 428c and the intermediate layer 428b), then through a Group II flow port 436 of the intermediate layer 428a (which provides less flow resistance than a filter trap 438 between the intermediate layer 428b and the intermediate layer 428a), then through the filter trap 438 between the intermediate layer 428a and the first end layer 426, and then through a Group I flow port 434 in the first end layer 426.

FIGS. 6C-E are generally directed to illustrating a representative fabrication technique that may be utilized for the filtering section 424. FIG. 6C illustrates part of the first end layer 426 with a Group I flow port 434 that extends entirely through the first end layer 426 (e.g., formed by patterning the first end layer 426). A sacrificial layer 440 thereafter may be deposited on the first end layer 426. The thickness of this sacrificial layer 440 will correspond with the height of the filter trap 438 between the first end layer 426 and the intermediate layer 428a. This sacrificial layer 440 will also be deposited in the Group I flow port 434 illustrated in FIG. 6C. This sacrificial layer 440 thereafter may then be patterned to define a plurality of structural interconnect apertures 442 that extend entirely through the sacrificial layer 440 (FIG. 6D). The intermediate layer 428a is then deposited on top of the sacrificial layer 440 and into the structural interconnect apertures 442 (FIG. 6E). This defines three structural interconnects 432 between the portion of the intermediate layer 428a and the first end layer 426 illustrated in FIG. 6E. Thereafter, the intermediate layer 428a may be patterned to define the Group II flow ports 436 that extend entirely through the intermediate layer 428a. This general process may be repeated for the various other layers in the stack 422.

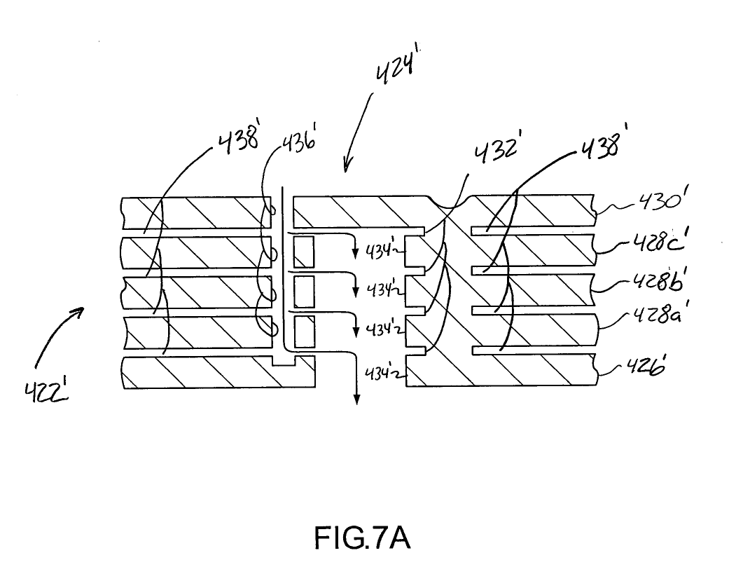

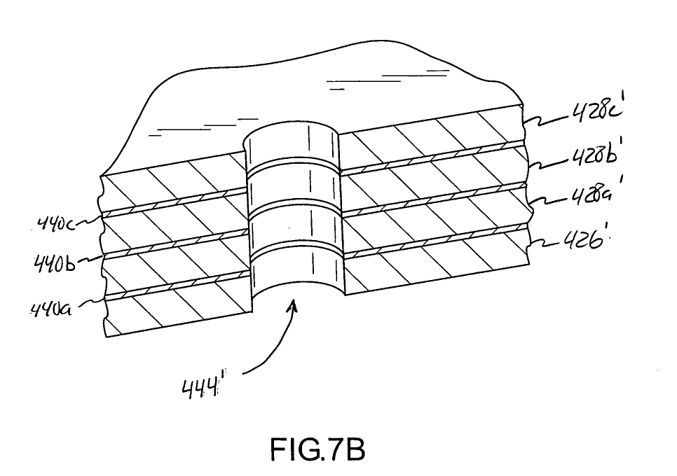

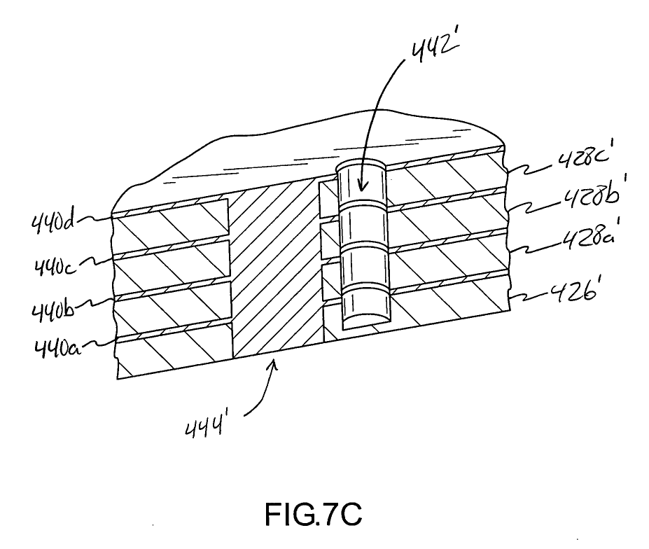

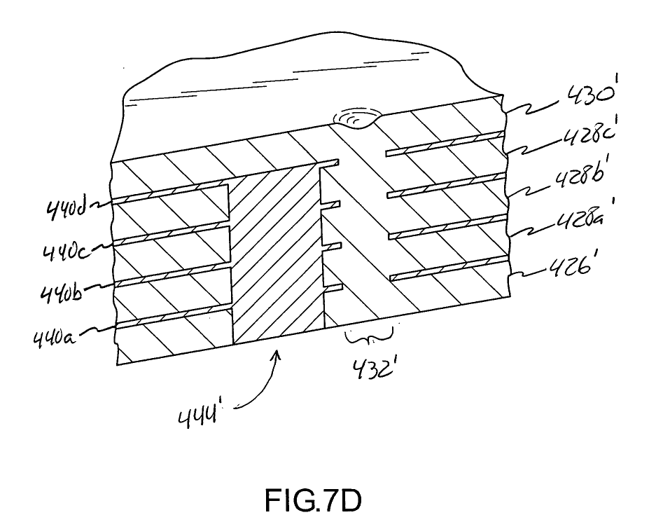

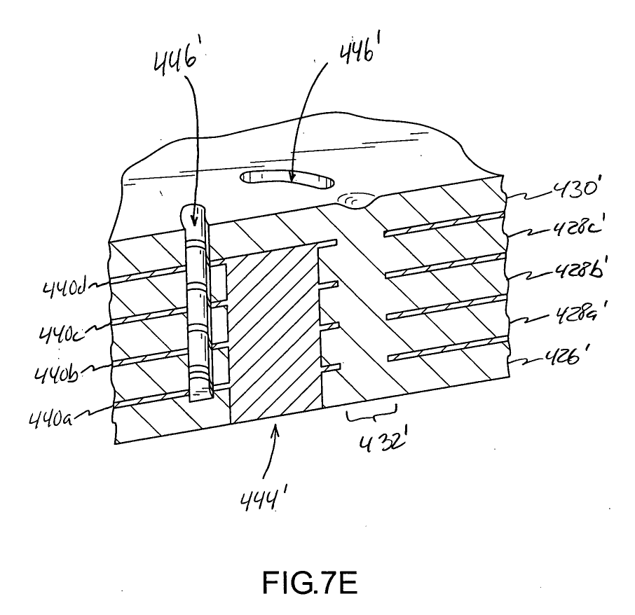

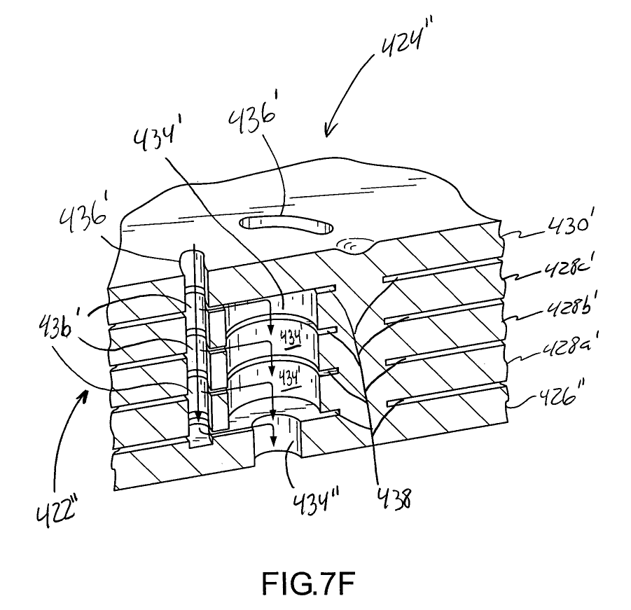

Another embodiment of a MEMS filtering section that includes a plurality of filter traps at each of a plurality of levels is illustrated in FIG. 7A, is identified by reference numeral 424′, and is a variation of the filtering section 424 of FIGS. 6A-B. Corresponding components between these two embodiments are identified the same reference numeral along with a single prime designation, and the discussion presented above with regard to these components for the filtering section 424 will remain equally applicable to the filtering section 424′ unless otherwise noted. The filtering section 424′ of FIG. 7A could be used in place of the filtering section 344 for the MEMS filter module 340 or any other MEMS flow module.

The filtering section 424′ includes a stack 422′ of a plurality of layers 426′, 428a′, 428b′, 428c′, and 430′ that are disposed at least substantially parallel relation and that are maintained in spaced relation by one or more structural interconnects 432′ that extend between adjacent layers. The primary distinction between the filtering section 424 of FIGS. 6A-6B and the filtering section 424′ of FIG. 7A is a result of the way in which these devices are fabricated. In this regard: the Group I flow ports 434′ through each of the layers 426′, 428a′, 428b′, and 428c′ are at least substantially the same size, in contrast to the filtering section 424 of FIGS. 6A-6B; the Group II flow ports 436′ through each of the layers 428a′, 428b′, 428c′, and 430′ are at least substantially the same size; the length of the filter traps 438′ between a corresponding Group II flow port 436′ and Group I flow port 434′ are at least substantially the same length, in contrast to the filtering section 424 of FIGS. 6A-6B.

FIGS. 7B-E are generally directed to illustrating a representative fabrication technique that may be utilized for the filtering section 424′. FIG. 7B illustrates that layer 426′ is formed, that a sacrificial layer 440a is thereafter formed on the layer 426′, that layer 428a′ is thereafter formed on the sacrificial layer 440b, that a sacrificial layer 440b is formed on layer 428a′, that layer 428b′ is thereafter formed on the sacrificial layer 440b, that a sacrificial layer 440c is thereafter formed on the layer 428b′, and that layer 428c′ is thereafter formed on the sacrificial layer 440c. Once this portion of the stack 422′ has been fabricated (all but the opposite end layer—second end layer 430′ in the illustrated embodiment), a single mask is used to simultaneously define a single Group I flow port aperture 444′ that extends through each of the layers 426′, 440a, 428a′, 440b, 428b′, 440c, and 428c′. The Group I flow port aperture 444′ through the layers 426′, 428a′, 428b′, and 428c′ will of course define a corresponding Group I flow port 434′.

Sacrificial layer 440d is then deposited on intermediate layer 428c′ and also into the Group I flow port aperture 444′ as illustrated in FIG. 7C. Although FIG. 7C illustrates sacrificial layer 440d completely filling Group I flow port aperture 444′, a central portion thereof may remain open (devoid of sacrificial material). In any case, a single mask is then used to simultaneously define a single structural interconnect aperture 442′ that extends through layers 440d, 428c′, 440c, 428b′, 440b, 428a′, 440a, and at least to the first end layer 426′ as also illustrated in FIG. 7C (e.g., partially into first end layer 426′ as shown; all the way through first end layer 426′ (not shown)). The second end layer 430′ is then deposited on the sacrificial layer 440d and into the structural interconnect aperture 442′ to structurally interconnect the layers 426′, 428a′, 428b′, 428c′, and 430′ as illustrated in FIG. 7D. This second end layer 430′ would also extend into any remaining aperture or void in the Group I flow port aperture 444′, with material of the sacrificial layer 440d being disposed entirely thereabout (e.g., when sacrificial layer 440d is removed, the resulting Group I flow port could be partially blocked by a structure extending from the second end layer 430′, but whose perimeter is entirely spaced from a wall that defines the perimeter of this Group I flow port). A single mask is then used to simultaneously define each of the Group II flow ports 436′ by forming (e.g., etching) a plurality of Group II flow port apertures 446′ that each extend down through each of the layers 430′, 440d, 428c′, 440c, 428b′, 440b, 428a′, and 440a, and possibly partially into the first end layer 426′, as shown in FIG. 7E. The remaining sacrificial material may then be removed to release the filtering section 424′. The Group II flow ports 436′ will then coincide with the corresponding portion of the Group II flow port aperture 446′ in each of the layers 430′, 428c′, 428b′, and 428a′.

Another embodiment of a MEMS filtering section that includes a plurality of filter traps at each of a plurality of levels is illustrated in FIG. 7F, is identified by reference numeral 424″, and is a variation of the filtering section 424′ of FIG. 7A. Corresponding components between these two embodiments are identified the same reference numeral, and the discussion presented above with regard to these components will remain equally applicable unless otherwise noted. The filtering section 424″ of FIG. 7F may be used in place of the filtering section 344 for the MEMS filter module 340 or any other MEMS flow module.

The only difference between the filtering section 424″ of FIG. 7F and the filtering section 424′ of FIG. 7A is that the Group I flow port 434″ through the first end layer 426″ of the stack 422″ is not the same size as the other Group I flow ports 434′. This difference is provided by a variation of the above-noted fabrication technique for the MEMS filter module 420′. Generally, the Group I flow port 434″ for the first end layer 426″ would be formed at least before forming the intermediate layer 428a over the sacrificial layer 440a. The remainder of the above-noted fabrication technique would thereafter be applicable.

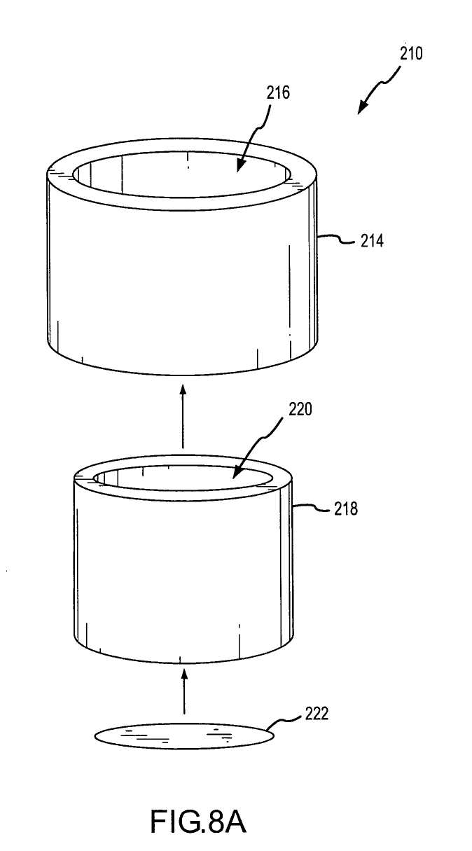

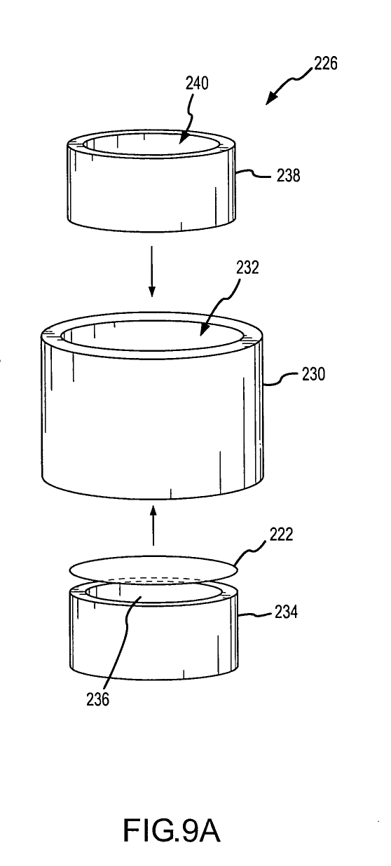

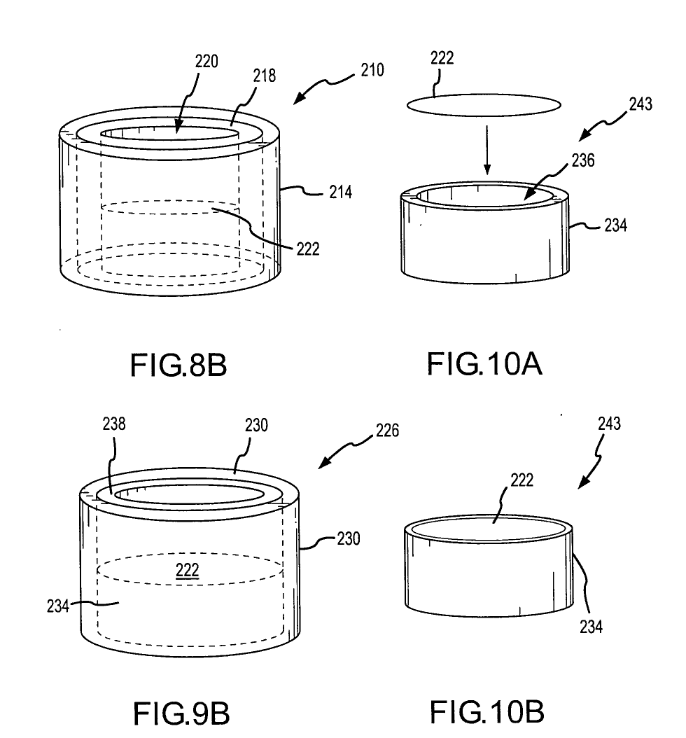

FIGS. 8A-B schematically represent one embodiment of a flow assembly 210 that may be used for any appropriate application (e.g., the flow assembly 210 may be disposed in a flow of any type, may be used to filter and/or control the flow of a fluid of any type, may be located in a conduit that fluidly interconnects multiple sources of any appropriate type (e.g., between multiple fluid or pressure sources (including where one is the environment), such as a man-made reservoir, a biological reservoir, the environment, or any other appropriate source), or any combination thereof). One example would be to dispose the flow assembly 210 in a conduit extending between the anterior chamber of an eye and a location that is exterior of the cornea of the eye. Another example would be to dispose the flow assembly 210 in a conduit extending between the anterior chamber of an eye and another location that is exterior of the sclera of the eye. Yet another example would be to dispose the flow assembly 210 in a conduit extending between the anterior chamber of an eye and another location within the eye (e.g., into Schlemm's canal) or body. In each of these examples, the conduit would provide an exit path for aqueous humor when installed for a glaucoma patient. That is, each of these examples may be viewed as a way of treating glaucoma or providing at least some degree of control of the intraocular pressure.

Components of the flow assembly 210 include an outer housing 214, an inner housing 218, and a MEMS flow module 222. Any of the MEMS filtering sections described herein may be used by the MEMS flow module 222, including without limitation the MEMS filtering sections 344 (FIGS. 3A-F), 394 (FIGS. 4A-B), 394′ (FIGS. 5A-B), 394″ (FIG. 5), 424 (FIGS. 6A-B), 424′ (FIG. 7A), or 424″ (FIG. 7F). The position of the MEMS flow module 222 and the inner housing 218 are at least generally depicted within the outer housing 214 in FIG. 8B to show the relative positioning of these components in the assembled condition—not to convey that the outer housing 214 needs to be in the form of a transparent structure. All details of the MEMS flow module 222 and the inner housing 218 are not necessarily illustrated in FIG. 8B.

The MEMS flow module 222 is only schematically represented in FIGS. 8A-B, and provides at least one of a filtering function and a pressure regulation function. The MEMS flow module 222 may be of any appropriate design, size, shape, and configuration, and further may be formed from any material or combination of materials that are appropriate for use by the relevant microfabrication technology. Any appropriate coating or combination of coatings may be applied to exposed surfaces of the MEMS flow module 222 as well. For instance, a coating may be applied to improve the biocompatibility of the MEMS flow module 222, to make the exposed surfaces of the MEMS flow module 222 more hydrophilic, to reduce the potential for the MEMS flow module 222 causing any bio-fouling, or any combination thereof. In one embodiment, a self-assembled monolayer coating (e.g., poly-ethylene-glycol) is applied in any appropriate manner (e.g., liquid or vapor phase, with vapor phase being the preferred technique) to all exposed surfaces of the MEMS flow module 222. The main requirement of the MEMS flow module 222 is that it is a MEMS device.

The primary function of the outer housing 214 and inner housing 218 is to provide structural integrity for the MEMS flow module 222 or to support the MEMS flow module 222, and further to protect the MEMS flow module 222. In this regard, the outer housing 214 and inner housing 218 each will typically be in the form of a structure that is sufficiently rigid to protect the MEMS flow module 222 from being damaged by the forces that reasonably could be expected to be exerted on the flow assembly 210 during its assembly, as well as during use of the flow assembly 210 in the application for which it was designed.

The inner housing 218 includes a hollow interior or a flow path 220 that extends through the inner housing 218 (between its opposite ends in the illustrated embodiment). The MEMS flow module 222 may be disposed within the flow path 220 through the inner housing 218 in any appropriate manner and at any appropriate location within the inner housing 218 (e.g., at any location so that the inner housing 218 is disposed about the MEMS flow module 222). Preferably, the MEMS flow module 222 is maintained in a fixed position relative to the inner housing 218. For instance, the MEMS flow module 222 may be attached or bonded to an inner sidewall or a flange formed on this inner sidewall of the inner housing 218, a press-fit could be provided between the inner housing 218 and the MEMS flow module 222, or a combination thereof The MEMS flow module 222 also could be attached to an end of the inner housing 218 in the manner of the embodiment of FIGS. 10A-B that will be discussed in more detail below.

The inner housing 218 is at least partially disposed within the outer housing 214 (thereby encompassing having the outer housing 214 being disposed about the inner housing 218 along the entire length of the inner housing 218, or only along a portion of the length of the inner housing 218). In this regard, the outer housing 214 includes a hollow interior 216 for receiving the inner housing 218, and possibly to provide other appropriate functionality (e.g., a flow path fluidly connected with the flow path 220 through the inner housing 218). The outer and inner sidewalls of the outer housing 214 may be cylindrical or of any other appropriate shape, as may be the outer and inner sidewalls of the inner housing 218. The inner housing 218 may be retained relative to the outer housing 214 in any appropriate manner. For instance, the inner housing 218 may be attached or bonded to an inner sidewall of the outer housing 214, a press-fit could be provided between the inner housing 218 and the outer housing 214, a shrink fit could be provided between the outer housing 214 and the inner housing 218, or a combination thereof.

The inner housing 218 is likewise only schematically represented in FIGS. 8A-B, and it may be of any appropriate shape/configuration, of any appropriate size, and formed from any material or combination of materials (e.g., polymethylmethacrylate (PMMA), ceramics, silicon, titanium, and other implantable metals and plastics). Typically its outer contour will be adapted to match the inner contour of the outer housing 214 in which it is at least partially disposed. In one embodiment, the illustrated cylindrical configuration for the inner housing 218 is achieved by cutting an appropriate length from hypodermic needle stock. The inner housing 218 also may be microfabricated into the desired/required shape (e.g., using at least part of a LIGA process). However, any way of making the inner housing 218 may be utilized. It should also be appreciated that the inner housing 218 may include one or more coatings as desired/required as well (e.g., an electroplated metal; a coating to improve the biocompatibility of the inner housing 218, to make the exposed surfaces of the inner housing 218 more hydrophilic, to reduce the potential for the inner housing 218 causing any bio-fouling, or any combination thereof). In one embodiment, a self-assembled monolayer coating (e.g., poly-ethylene-glycol) is applied in any appropriate manner (e.g., liquid or vapor phase, with vapor phase being the preferred technique) to all exposed surfaces of the inner housing 218.

The outer housing 214 likewise is only schematically represented in FIGS. 8A-B, and it may be of any appropriate shape/configuration, of any appropriate size, and formed from any material or combination of materials (e.g., polymethylmethacrylate (PMMA), ceramics, silicon, titanium, and other implantable metals and plastics). Typically its outer contour will be adapted to match the inner contour of the housing or conduit in which it is at least partially disposed or otherwise mounted. The outer housing 214 also may be microfabricated into the desired/required shape (e.g., using at least part of a LIGA process). However, any way of making the outer housing 214 may be utilized. It should also be appreciated that the outer housing 214 may include one or more coatings as desired/required as well (e.g., an electroplated metal; a coating to improve the biocompatibility of the outer housing 214, to make the exposed surfaces of the outer housing 214 more hydrophilic, to reduce the potential for the outer housing 214 causing any bio-fouling, or any combination thereof). In one embodiment, a self-assembled monolayer coating (e.g., poly-ethylene-glycol) is applied in any appropriate manner (e.g., liquid or vapor phase, with vapor phase being the preferred technique) to all exposed surfaces of the outer housing 214.

Another embodiment of a flow assembly is illustrated in FIGS. 9A-B (only schematic representations), and is identified by reference numeral 226. The flow assembly 226 may be used for any appropriate application (e.g., the flow assembly 226 may be disposed in a flow of any type, may be used to filter and/or control the flow of a fluid of any type, may be located in a conduit that fluidly interconnects multiple sources of any appropriate type (e.g., multiple fluid or pressure sources (including where one is the environment), such as a man-made reservoir, a biological reservoir, the environment, or any other appropriate source), or any combination thereof). The above-noted applications for the flow assembly 210 are equally applicable to the flow assembly to 226. The types of coatings discussed above in relation to the flow assembly 210 may be used by the flow assembly 226 as well.

Components of the flow assembly 226 include an outer housing 230, a first inner housing 234, a second inner housing 238, and the MEMS flow module 222. The MEMS flow module 222 and the inner housings 234, 238 are at least generally depicted within the outer housing 230 in FIG. 9B to show the relative positioning of these components in the assembled condition—not to convey that the outer housing 230 needs to be in the form of a transparent structure. All details of the MEMS flow module 222 and the inner housings 234, 238 are not necessarily illustrated in FIG. 9B.

The primary function of the outer housing 230, first inner housing 234, and second inner housing 238 is to provide structural integrity for the MEMS flow module 222 or to support the MEMS flow module 222, and further to protect the MEMS flow module 222. In this regard, the outer housing 230, first inner housing 234, and second inner housing 238 each will typically be in the form of a structure that is sufficiently rigid to protect the MEMS flow module 222 from being damaged by the forces that reasonably could be expected to be exerted on the flow assembly 226 during its assembly, as well as during use of the flow assembly 226 in the application for which it was designed.

The first inner housing 234 includes a hollow interior or a flow path 236 that extends through the first inner housing 234. Similarly, the second inner housing 238 includes a hollow interior or a flow path 240 that extends through the second inner housing 238. The first inner housing 234 and the second inner housing 238 are disposed in end-to-end relation, with the MEMS flow module 222 being disposed between adjacent ends of the first inner housing 234 and the second inner housing 238. As such, a flow progressing through the first flow path 236 to the second flow path 240, or vice versa, passes through the MEMS flow module 222.

Preferably, the MEMS flow module 222 is maintained in a fixed position relative to each inner housing 234, 238, and its perimeter does not protrude beyond the adjacent sidewalls of the inner housings 234, 238 in the assembled and joined condition. For instance, the MEMS flow module 222 may be bonded to at least one of, but more preferably both of, the first inner housing 234 (more specifically one end thereof) and the second inner housing 238 (more specifically one end thereof) to provide structural integrity for the MEMS flow module 222 (e.g., using cyanoacrylic esters, thermal bonding, UV-curable epoxies, or other epoxies). Another option would be to fix the position the MEMS flow module 222 in the flow assembly 226 at least primarily by fixing the position of each of the inner housings 234, 238 relative to the outer housing 230 (i.e., the MEMS flow module 222 need not necessarily be bonded to either of the housings 234, 238). In one embodiment, an elastomeric material may be disposed between the MEMS flow module 222 and the first inner housing 234 to allow the first inner housing 234 with the MEMS flow module 222 disposed thereon to be pushed into the outer housing 230 (e.g., the elastomeric material is sufficiently “tacky” to at least temporarily retain the MEMS flow module 222 in position relative to the first inner housing 234 while being installed in the outer housing 230). The second inner housing 238 also may be pushed into the outer housing 230 (before, but more likely after, the first inner housing 234 is disposed in the outer housing 230) to “sandwich” the MEMS flow module 222 between the inner housings 234, 238 at a location that is within the outer housing 230 (i.e., such that the outer housing 230 is disposed about MEMS flow module 222). The MEMS flow module 222 would typically be contacted by both the first inner housing 234 and the second inner housing 238 when disposed within the outer housing 230. Fixing the position of each of the first inner housing 234 and the second inner housing 238 relative to the outer housing 230 will thereby in effect fix the position of the MEMS flow module 222 relative to the outer housing 230. Both the first inner housing 234 and second inner housing 238 are at least partially disposed within the outer housing 230 (thereby encompassing the outer housing 230 being disposed about either or both housings 234, 238 along the entire length thereof, or only along a portion of the length of thereof), again with the MEMS flow module 222 being located between the adjacent ends of the first inner housing 234 and the second inner housing 238. In this regard, the outer housing 230 includes a hollow interior 232 for receiving at least part of the first inner housing 234, at least part of the second inner housing 238, and the MEMS flow module 222 disposed therebetween, and possibly to provide other appropriate functionality (e.g., a flow path fluidly connected with the flow paths 236, 240 through the first and second inner housings 234, 238, respectively). The outer and inner sidewalls of the outer housing 230 may be cylindrical or of any other appropriate shape, as may be the outer and inner sidewalls of the inner housings 234, 238. Both the first inner housing 234 and the second inner housing 238 may be secured to the outer housing 230 in any appropriate manner, including in the manner discussed above in relation to the inner housing 218 and the outer housing 214 of the embodiment of FIGS. 8A-B.

Each inner housing 234, 238 is likewise only schematically represented in FIGS. 9A-B, and each may be of any appropriate shape/configuration, of any appropriate size, and formed from any material or combination of materials in the same manner as the inner housing 218 of the embodiment of FIGS. 7-8. Typically the outer contour of both housings 234, 238 will be adapted to match the inner contour of the outer housing 230 in which they are at least partially disposed. In one embodiment, the illustrated cylindrical configuration for the inner housings 234, 238 is achieved by cutting an appropriate length from hypodermic needle stock. The inner housings 234, 238 each also may be microfabricated into the desired/required shape (e.g., using at least part of a LIGA process). However, any way of making the inner housings 234, 238 may be utilized. It should also be appreciated that the inner housings 234, 238 may include one or more coatings as desired/required as well in accordance with the foregoing.

The outer housing 230 is likewise only schematically represented in FIGS. 9A-B, and it may be of any appropriate shape/configuration, of any appropriate size, and formed from any material or combination of materials in the same manner as the outer housing 214 of the embodiment of FIGS. 8A-B. Typically the outer contour of the outer housing 230 will be adapted to match the inner contour of the housing or conduit in which it is at least partially disposed or otherwise mounted. The outer housing 230 may be microfabricated into the desired/required shape (e.g., using at least part of a LIGA process). However, any way of making the outer housing 230 may be utilized. It should also be appreciated that the outer housing 230 may include one or more coatings as desired/required in accordance with the foregoing.

Another embodiment of a flow assembly is illustrated in FIGS. 10A-B (only schematic representations), and is identified by reference numeral 243. The flow assembly 243 may be used for any appropriate application (e.g., the flow assembly 243 may be disposed in a flow of any type, may be used to filter and/or control the flow of a fluid of any type, may be located in a conduit that fluidly interconnects multiple sources of any appropriate type (e.g., between multiple fluid or pressure sources, such as a man-made reservoir, a biological reservoir, the environment, or any other appropriate source), or any combination thereof). Components of the flow assembly 243 include the above-noted housing 234 and the MEMS flow module 222 from the embodiment of FIGS. 9A-B. In the case of the flow assembly 243, the MEMS flow module 222 is attached or bonded to one end of the housing 234 (e.g., using cyanoacrylic esters, thermal bonding, UV-curable epoxies, or other epoxies). The flow assembly 243 may be disposed within an outer housing in the manner of the embodiments of FIGS. 8A-9B, or could be used “as is.” The above-noted applications for the flow assembly 210 are equally applicable to the flow assembly 243. The types of coatings discussed above in relation to the flow assembly 210 may be used by the flow assembly 243 as well.

One particularly desirable application for the flow assemblies 210, 226, and 243 of FIGS. 8A-10B, as discussed above, is to regulate pressure within the anterior chamber of an eye. That is, they may be disposed in an exit path through which aqueous humor travels to treat a glaucoma patient. Preferably, the flow assemblies 210, 226, 243 each provide a bacterial filtration function to reduce the potential for developing an infection within the eye. Although the various housings and MEMS flow modules used by the flow assemblies 210, 226, and 243 each may be of any appropriate color, it may be desirable for the color to be selected so as to “blend in” with the eye to at least some extent.

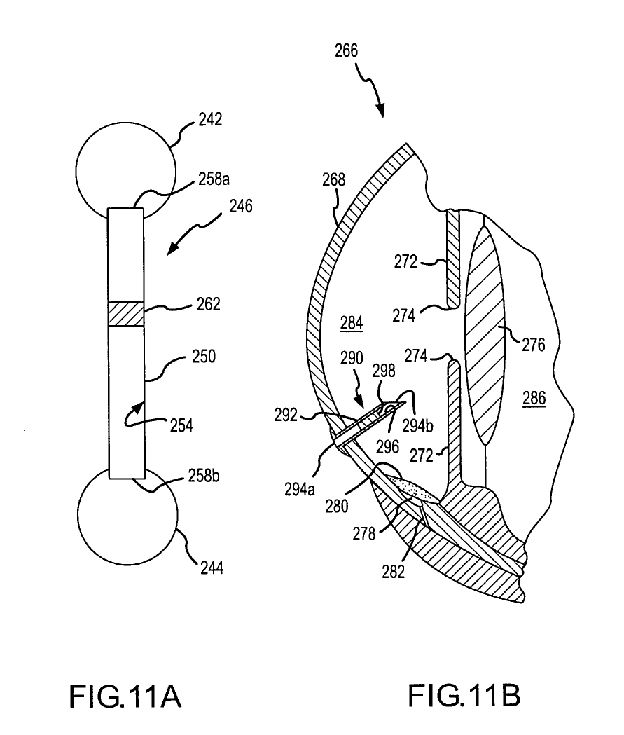

An example of the above-noted application is schematically illustrated in FIG. 11A. Here, an anterior chamber 242 of a patient's eye (or other body region for that matter—a first body region) is fluidly interconnected with an appropriate drainage area 244 by an implant, shunt, or drainage device 246 (a “glaucoma drainage device” for the specifically noted case). The drainage area 244 may be any appropriate location, such as externally of the eye (e.g., on an exterior surface of the cornea), within the eye (e.g., Schlemm's canal), or within the patient's body in general (a second body region).

Generally, the drainage device 246 includes a conduit 250 having a pair of ends 258a, 258b, with a flow path 254 extending therebetween. The size, shape, and configuration of the conduit 250 may be adapted as desired/required, including to accommodate the specific drainage area 244 being used. Representative configurations for the conduit 250 are disclosed in U.S. Patent Application Publication No. 2003/0212383, as well as U.S. Pat. Nos. 3,788,327; 5,743,868; 5,807,302; 6,626,858; 6,638,239; 6,533,768; 6,595,945; 6,666,841; and 6,736,791, the entire disclosures of which are incorporated by reference in their entirety herein.

A flow module 262 is disposed within the flow path 254 of the conduit 250. All flow leaving the anterior chamber 242 through the implant 246 is thereby directed through the flow module 262. Similarly, any flow from the drainage area 244 into the implant 246 will have to pass through the flow module 262. The flow module 262 may be retained within the conduit 250 in any appropriate manner and at any appropriate location (e.g., it could be disposed on either end 258a, 258b, or any intermediate location therebetween). The flow module 262 may be integrated using one or more housings (e.g., in the manner of any of the flow assemblies 210, 226, or 243 (FIGS. 8A-10B)). Alternatively, the flow module 262 could be directly disposed within the conduit 250 as shown. Any appropriate coating may be applied to at least those surfaces of the drainage device 246 that would be exposed to biological material/fluids, including without limitation a coating that improves biocompatibility, that makes such surfaces more hydrophilic, and/or that reduces the potential for bio-fouling. In one embodiment, a self- assembled monolayer coating (e.g., poly-ethylene-glycol) is applied in any appropriate manner (e.g., liquid or vapor phase, with vapor phase being the preferred technique) to the noted surfaces.

FIG. 11B illustrates a representative embodiment in accordance with FIG. 11A. Various portions of the eye 266 are identified in FIG. 11B, including the cornea 268, iris 272, pupil 274, lens 276, anterior chamber 284, vitreous body 286, Schlemm's canal 278, trabecular meshwork 280, and aqueous veins 282. Here, a drainage device, implant, or shunt 290 having an appropriately-shaped conduit 292 is directed through the cornea 268. The conduit 292 may be in any appropriate form, but will typically include at least a pair of ends 294a, 294b, as well as a flow path 296 extending therebetween. End 294a is disposed on the exterior surface of the cornea 268, while end 294b is disposed within the anterior chamber 284 of the eye 266.

A flow module 298 is disposed within the flow path 296 of the conduit 292. All flow leaving the anterior chamber 284 through the drainage device 290 is thereby directed through the flow module 298. Similarly, any flow from the environment back into the drainage device 290 will have to pass through the flow module 298 as well. Preferably, the flow module 298 provides a bacterial filtration function to reduce the potential for developing an infection within the eye when using the drainage device 290. The flow module 298 may be retained within the conduit 292 in any appropriate manner and at any appropriate location (e.g., it could be disposed on either end 294a, 294b, or any an intermediate location therebetween). The flow module 298 may be integrated using one or more housings (e.g., in the manner of any of the flow assemblies 210, 226, or 243 (FIGS. 8A-10B)). Alternatively, the flow module 298 could be directly disposed within the conduit 292. Any appropriate coating may be applied to at least those surfaces of the drainage device 290 that would be exposed to biological material/fluids, including without limitation a coating that improves biocompatibility, that makes such surfaces more hydrophilic, and/or that reduces the potential for bio-fouling. In one embodiment, a self-assembled monolayer coating (e.g., poly-ethylene-glycol) is applied in any appropriate manner (e.g., liquid or vapor phase, with vapor phase being the preferred technique) to the noted surfaces.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such, or other embodiments and with various modifications required by the particular application(s) or use(s) of the present invention. It is intended that the appended claims be construed to include alternative embodiments to the extent permitted by the prior art.

What is claimed is:

1. A MEMS filter module, comprising:
a stack of a plurality of layers that are structurally interconnected, wherein said plurality of layers are stacked in a first dimension, wherein said stack comprises a first filtering section, wherein each layer of said plurality of layers comprises at least one flow port within said first filtering section, and wherein each said flow port extends completely through its corresponding said layer;
a first filter trap within said first filtering section, wherein all flow through said first filter trap is in a second dimension that is different from said first dimension; and
a second filter trap within said first filtering section, wherein all flow through said second filter trap is also in said second dimension, wherein said first and second filter traps are disposed at different locations within said first dimension, and wherein said first and second filter traps provide a greater flow resistance than each individual said flow port.

2. The MEMS filter module of claim 1, wherein said plurality of layers comprises at least three separate layers.

3. The MEMS filter module of claim 1, wherein said plurality of layers comprises first, second, and third layers, wherein said second layer is located between said first and third layers, wherein said first filter trap is associated with a flow path between said first and second layers, and wherein said second filter trap is associated with a flow path between said second and third layers.

4. The MEMS filter module of claim 1, wherein each said layer of said plurality of layers comprises a thickness within a range of about 1 micron to about 3 microns.

5. The MEMS filter module of claim 1, wherein each said layer of said plurality of layers is associated with a different fabrication level.

6. The MEMS filter module of claim 1, wherein said first and second filter traps each have a height dimension of no more than about 0.4 microns, wherein said height dimension is orthogonal to said second dimension.

7. The MEMS filter module of claim 1, wherein said second dimension is at least substantially orthogonal to said first dimension.

8. The MEMS filter module of claim 1, wherein a flow through said first and second filter traps is in a common direction within said second dimension.

9. The MEMS filter module of claim 1, wherein a flow through said first and second filter traps are in opposite directions within said second dimension.

10. The MEMS filter module of claim 1, wherein at least one of said first and second filter traps is annular.

11. The MEMS filter module of claim 1, wherein each of said first and second filter traps is annular.

12. The MEMS filter module of claim 1, wherein said first and second filter traps are of a common configuration.

13. The MEMS filter module of claim 1, wherein said first and second filter traps are of a different configuration.

14. The MEMS filter module of claim 1, wherein said plurality of layers comprises first and second layers, wherein said first filtering section further comprises a filtering wall that extends from said first layer toward said second layer, wherein a space between a distal end of said filtering wall and said second layer defines said first filter trap.

15. The MEMS filter module of claim 14, wherein said plurality of layers comprises a first adjacent pair of layers, wherein a maximum spacing between first and second members of said first adjacent pair of layers within said first filtering section defines said second filter trap.

16. The MEMS filter module of claim 14, wherein said plurality of layers comprises a first adjacent pair of layers, wherein at least substantially planar surfaces of first and second members of said first adjacent pair of layers that face each other within said first filtering section define said first filter trap.

17. The MEMS filter module of claim 1, wherein said plurality of layers comprises first and second layers, wherein a maximum spacing between said first and second layers within said first filtering section defines said first filter trap.

18. The MEMS filter module of claim 1, wherein said plurality of layers comprises first and second layers, wherein at least substantially planar first and second surfaces of said first and second layers, respectively, are spaced from each other within said first filtering section and collectively define said first filter trap, and wherein said first and second surfaces are of a common size.

19. The MEMS filter module claim 1, wherein said first filtering section further comprises a plurality of filter traps that in turn comprise said first and second filter traps, wherein a cavity of a constant, fixed height exists between each adjacent pair of said plurality of layers, and defines a corresponding said filter trap.

20. The MEMS filter module of claim 1, further comprising a plurality of said first filtering sections.

21. The MEMS filter module of claim 1, further comprising at least one annular seal between each adjacent pair of layers of said plurality of layers, wherein said first filtering section is located inwardly of each said at least one annular seal.

22. An implant associated with a first body region and that comprises said MEMS filter module of claim 1 and a conduit, wherein said conduit comprises a flow path that is adapted to fluidly interconnect with the first body region, and wherein said MEMS filter module is disposed in said flow path.

23. The implant of claim 22, further comprising at least one housing, wherein said at least one housing is disposed within said conduit, and wherein said MEMS filter module interfaces with said at least one housing.

24. A drainage device installable in a human eye and comprising said MEMS filter module of claim 1 and a conduit, wherein said conduit comprises a flow path that is adapted to fluidly interconnect with an anterior chamber of the human eye when said drainage device is installed, and wherein said MEMS filter module is disposed in said flow path.

25. The MEMS filter module of claim 1, wherein said first filtering section further comprises a plurality of filter traps that in turn comprises said first and second filter traps, wherein said plurality of layers comprises first, second, and third layers, wherein said second layer is located between said first and third layers, wherein said at least one flow port for each of s said first, second, and third layers is selected from the group consisting of at least one of a Group I flow port, at least one of a Group II flow port, or any combination thereof, wherein each said Group I and Group II flow port has a smaller flow resistance than either of said first and second filter traps, wherein said first and second layers each comprise a first said Group I flow port, wherein said second and third layers each comprise a first said Group II flow port, wherein said first layer is devoid of any said Group II flow port, wherein said third layer is devoid of any said Group I flow port, wherein all flow between any said Group I flow port and any said Group II flow port must pass through at least one of said plurality of filter traps.

26. The MEMS filter module of claim 1, wherein said first filtering section comprises a plurality of filter traps that in turn comprises said first and second filter traps, wherein said plurality of layers comprises a first and second end layers and a first intermediate layer, wherein said first end layer comprises at least one Group I flow port and is devoid of any Group II flow port, wherein said second end layer comprises at least one said Group II flow port and is devoid of any said Group II flow port, wherein said first intermediate layer comprises at least one said Group I flow port and at least one said Group II flow port, wherein each said Group I and Group II flow port has a smaller flow resistance than each of said plurality of filter traps, and wherein all flow between any said Group I flow port of said first end layer and any said Group II flow port of said second end layer is required to pass through at least one of said plurality of filter traps.

27. The MEMS filter module of claim 1, wherein said first filtering section comprises a plurality of filter traps that in turn comprises said first and second filter traps, wherein said plurality of layers comprises a first and second end layers and at a first intermediate layer, wherein said first and second end layers each comprises at least one Group I flow port and are devoid of any Group II flow port, wherein said first intermediate layer comprises at least one said Group I flow port and at least one said Group II flow port, wherein each said Group I and Group II flow port has a smaller flow resistance than each of said plurality of filter traps, and wherein all flow between any said Group I flow port of said first end layer and any said Group I flow port of said second end layer is required to pass through at least one of said plurality of filter traps at each of two different locations within said stack in said first dimension.

28. The MEMS filter module of claim 27, wherein a flow through a first of said at least two of said plurality of filter traps is in a first direction and a flow through a second of said at least two of said plurality of filter traps is in a second direction that is different from said first direction.

29. The MEMS filter module of claim 1, wherein said first filtering section comprises a plurality of filter traps that in turn comprises said first and second filter traps, wherein said plurality of layers comprises a first and second end layers and at a first intermediate layer, wherein said first and second end layers each comprises at least one Group II flow port and are devoid of any Group I flow port, wherein said first intermediate layer comprises at least one said Group I flow port and at least one said Group II flow port, wherein each said Group I and Group II flow port has a smaller flow resistance than each of said plurality of filter traps, and wherein all flow between any said Group II flow port of said first end layer and any said Group II flow port of said second end layer is required to pass through at least one of said plurality of filter traps at each of two different locations within said stack in said first dimension.

30. The MEMS filter module of claim 1, wherein said plurality of layers comprises a first pair of adjacent layers and a second pair of adjacent layers, wherein said at least one flow port for each layer of said first and second pair of adjacent layers is selected from the group consisting of at least one Group I flow port, at least one Group II flow port, or any combination thereof, wherein each said Group I and Group II flow port has a smaller flow resistance than either of said first and second filter traps, wherein an at least substantially constant spacing exists between each layer of said first pair of adjacent layers and defines said first filter trap, wherein an at least substantially constant spacing exists between each layer of said second pair of adjacent layers and defines said second filter trap.

31. The MEMS filter module of claim 30, wherein said first and second filter traps are of a common length.

32. The MEMS filter module of claim 30, wherein said first and second filter traps are of a different length.

33. The MEMS filter module of claim 1, wherein said plurality of layers comprises a first and second end layers, at least one intermediate layer, and a plurality of filter traps that comprises said first and second filter traps, wherein said at least one flow port for each of said plurality of layers is selected from the group consisting of at least one Group I flow port, at least one Group II flow port, or any combination thereof, wherein each said Group I and Group II flow port has a smaller flow resistance than either of said first and second filter traps, wherein an at least substantially constant spacing exists between each adjacent pair of layers of said plurality of layers and defines a corresponding said filter trap, wherein said first end layer comprises at least one said Group II flow port and is devoid of any said Group I flow port, wherein each said intermediate layer comprises a plurality of said Group II flow ports and at least one said Group I flow port, and wherein said second end layer comprises a first said Group I flow port and is devoid of any said Group II flow port.

34. The MEMS filter module claim 33, wherein each said Group II flow port of said first end layer is axially aligned with one said Group II flow port from each said intermediate layer, and wherein said first said Group I flow port of each said intermediate layer and said first said Group I flow port of said second end layer are axially aligned.

35. The MEMS filter module of claim 34, wherein a size of said Group II flow ports progressively decreases from layer to layer progressing in a said second end layer, and wherein a size of said first said Group I flow ports progressively decreases from layer to layer progressing in a direction of said second end layer.

36. The MEMS filter module of claim 34, wherein each said Group II flow port is of the same size, and wherein each said first said Group I flow port is of the same size.

37. The MEMS filter module of claim 1, wherein said plurality of layers comprises first, second, and third layers, wherein said second layer is located between said first and third layers, wherein said at least one flow port for each of said first, second, and third layers is selected from the group consisting of at least one Group I flow port, at least one Group II flow port, or any combination thereof, wherein each said Group I and Group II flow port has a smaller flow resistance than either of said first and second filter traps, wherein said first and second layers each comprise a first said Group I flow port, wherein said second and third layers each comprise a first said Group II flow port, wherein said first layer is devoid of any said Group II flow port, wherein said third layer is devoid of any said Group I flow port, wherein a flow path between said first said Group II flow port of said third layer and said first said Group I flow port of said second layer includes said second filter trap, wherein a flow path between said first said Group I flow port of said second layer and said first said Group I flow port of said first layer excludes said first and second filter traps, wherein a flow path between said first said Group II flow port of said third layer and said first said Group II flow port of said second layer excludes said first and second filter traps, and wherein a flow path between said first said Group II flow port of said second layer and said first said Group I flow port of said first layer includes said first filter trap.

38. The MEMS filter module of claim 37, wherein said stack further comprises a first intermediate layer disposed between said first and second layers, wherein said at least one flow port for said first intermediate layer comprises a second said Group I flow port and a second said Group II flow port, wherein said MEMS filter module further comprises a first intermediate filter trap, wherein all flow through said first intermediate filter trap is in said second dimension, wherein said first intermediate filter trap provides a greater flow resistance that each individual said flow port, wherein a flow path between said second Group I flow port of said first intermediate layer and said first said Group I flow port of said first layer excludes said first filter trap, said second filter trap, and said first intermediate filter trap, and wherein a flow path between said second said Group II flow port of first intermediate layer and said first said Group I flow port of said first layer includes said first intermediate filter trap.

39. The MEMS filter module of claim 37, wherein said stack further comprises a first sub layer, wherein said first layer is located between said second layer and said first sub layer, wherein said at least one flow port for said first sub layer comprises a second said Group I flow port and a second said Group II flow port, wherein said MEMS filter module further comprises a first sub filter trap, wherein all flow through said first sub filter trap is in said second dimension, wherein said first sub filter trap provides a greater flow resistance that each individual said flow port, wherein a flow path between said first said Group I flow port of said first layer and said second said Group I flow port of said first sub layer excludes said first filter trap, said second filter trap, and said first sub filter trap, and wherein a flow path between said first said Group I flow port of said first layer and said second said Group II flow port of said first sub layer includes said first sub filter trap.

40. The MEMS filter module of claim 39, wherein said stack further comprises a second sub layer, wherein said first sub layer is located between said first layer and said second sub layer, wherein said at least one flow port for said second sub layer comprises a third said Group II flow port, wherein said second sub layer is devoid of any said Group I flow port, wherein said MEMS filter module further comprises a second sub filter trap, wherein all flow through each said second sub filter trap is in said second dimension, wherein said second sub filter trap provides a greater flow resistance than each individual said flow port, wherein a flow path between said second said Group II flow port of said first sub layer and said third said Group II flow port of second sub layer excludes said first flow trap, said second flow trap, said first sub filter trap, and said second sub filter trap, and wherein a flow path between said second said Group I flow port of first sub layer and said third said Group II flow port of said second sub layer includes said second sub filter trap.

41. The MEMS filter module of claim 1, wherein said plurality of layers comprises first, second, third, and fourth layers, wherein said third layer is located between said fourth layer and said second layer, wherein said second layer is located between said third layer and said first layer, wherein said at least one flow port for each of said first, second, and third layers is selected from the group consisting of at least one Group I flow port, at least one Group II flow port, or any combination thereof, wherein said MEMS filter module further comprises a third filter trap within said first filtering section that is disposed at a different elevation than each of said first and second filter traps, wherein all flow through said third filter trap is in said second dimension, wherein each said Group I and Group II flow port has a smaller flow resistance than either of said first, second filter, and third traps, wherein said fourth layer comprises a plurality of fourth said Group II flow ports and is devoid of any said Group I flow port, wherein said third layer comprises a plurality of third said Group II flow ports and a third said Group I flow port, wherein said second layer comprises a plurality of second said Group II flow ports and a second said Group I flow port, wherein said first layer comprises a first said Group I flow port and is devoid of any said Group II flow port, wherein said third filter trap is disposed in a flow path between each of said fourth said Group II flow ports and said third said Group I flow port, wherein said second filter trap is disposed in a flow path between each of said third said Group II flow ports and said second said Group I flow port, wherein said first filter trap is disposed in a flow path between each of said second said Group II flow ports and said first said Group I flow port, and wherein all flow between said plurality of fourth said Group II flow ports and said first said Group I flow port must pass through at least one of said first, second, and third filter traps.

42. The MEMS filter module claim 41, wherein each said fourth said Group II flow port is axially aligned with one said third said Group II flow port and one said second said Group to flow port, and wherein said third said Group I flow port, said second said Group I flow port, and said first said Group I flow port are axially aligned.

43. The MEMS filter module claim 42, wherein said plurality of third said Group II flow ports are disposed about said third said Group I flow port, and wherein said plurality of second said Group II flow ports are disposed about said second said Group I flow port.

44. The MEMS filter module claim 41, wherein said plurality of third said Group II flow ports are disposed about said third said Group I flow port, and wherein said plurality of second said Group II flow ports are disposed about said second said Group I flow port.

45. The MEMS filter module of claim 1, wherein said plurality of layers comprises first, second, third, fourth, and fifth layers, wherein said fourth layer is located between said fifth layer and said third layer, wherein said third layer is located between said fourth layer and said second layer, wherein said second layer is located between said third layer and said first layer, wherein said at least one flow port for each of said first, second, third, fourth, and fifth layers is selected from the group consisting of at least one Group I flow port, at least one Group II flow port, or any combination thereof, wherein said MEMS filter module further comprises third and fourth filter traps within said first filtering section, wherein all flow through each of said third and fourth filter traps is in said second dimension, wherein each of said first, second, third, and fourth filter traps are disposed at different elevations within said stack, wherein each said Group I and Group II flow port has a smaller flow resistance than either of said first, second filter, third, and fourth traps, wherein said fifth layer comprises a plurality of fifth said Group II flow ports and is devoid of any said Group I flow port, wherein said fourth layer comprises a plurality of fourth said Group II flow ports and a fourth said Group I flow port, wherein said third layer comprises a plurality of third said Group II flow ports and a third said Group I flow port, wherein said second layer comprises a plurality of second said Group II flow ports and a second said Group I flow port, wherein said first layer comprises a first said Group I flow port and is devoid of any said Group II flow port, wherein said fourth filter trap is disposed in a flow path between each of said fifth said Group II flow ports and said fourth said Group I flow port, wherein said third filter trap is disposed in a flow path between each of said fourth said Group II flow ports and said third said Group I flow port, wherein said second filter trap is disposed in a flow path between each of said third said Group II flow ports and said second said Group I flow port, wherein said first filter trap is disposed in a flow path between each of said second said Group II flow ports and said first Group I flow port, and wherein all flow between said plurality of fifth said Group II flow ports and said first Group I flow port must pass through at least one of said first, second, third, and fourth filter traps.

46. The MEMS filter module claim 45, wherein each said fifth said Group II flow port is axially aligned with one said fourth said Group II flow port, one said third said Group II flow port, and one said second said Group to flow port, and wherein said fourth said Group I flow port, said third said Group I flow port, said second said Group I flow port, and said first said Group I flow port are axially aligned.

47. The MEMS filter module claim 46, wherein said plurality of fourth said Group II flow ports are disposed about said fourth said Group I flow port, wherein said plurality of third said Group II flow ports are disposed about said third said Group I flow port, and wherein said plurality of second said Group II flow ports are disposed about said second said Group I flow port.

48. The MEMS filter module claim 45, wherein said plurality of fourth said Group II flow ports are disposed about said fourth said Group I flow port, wherein said plurality of third said Group II flow ports are disposed about said third said Group I flow port, and wherein said plurality of second said Group II flow ports are disposed about said second said Group I flow port.

49. The MEMS filter module of claim 1, wherein said plurality of layers comprises first, second, third, fourth, and fifth layers, wherein said fourth layer is located between said fifth layer and said third layer, wherein said third layer is located between said fourth layer and said second layer, wherein said second layer is located between said third layer and said first layer, wherein said at least one flow port for each of said first, second, third, fourth, and fifth layers is selected from the group consisting of at least one Group I flow port, at least one Group II flow port, or any combination thereof, wherein said MEMS filter module further comprises third and fourth filter traps within said first filtering section, wherein all flow through each of said third and fourth filter traps is in said second dimension, wherein each of said first, second, third, and fourth filter traps are disposed at different elevations within said stack, wherein each said Group I and Group II flow port has a smaller flow resistance than either of said first, second filter, third, and fourth traps, wherein said fifth layer comprises a plurality of fifth said Group II flow ports and is devoid of any said Group I flow port, wherein said fourth layer comprises a plurality of fourth said Group II flow ports and a fourth said Group I flow port, wherein said third layer comprises a third said Group I flow port and is devoid of any said Group II flow port, wherein said second layer comprises a plurality of second said Group II flow ports and a second Group I flow port, wherein said first layer comprises a plurality of first said Group II flow ports and is devoid of any said Group I flow port, wherein said fourth filter trap is disposed in a flow path between each of said fifth said Group II flow ports and said fourth said Group I flow port, wherein said third filter trap is disposed in a flow path between each of said fourth said Group II flow ports and said third said Group I flow port, wherein said second filter trap is disposed in a flow path between said third Group I flow port and each of said second said Group II flow ports, wherein said first filter trap is disposed in a flow path between said second said Group I flow port and each of said first said Group II flow ports, and wherein all flow between said plurality of fifth said Group II flow ports and said plurality of first said Group II flow ports must pass through at least one of said third and fourth filter traps and also must pass through at least one of said first and said second filter traps.

50. The MEMS filter module claim 49, wherein each said fifth said Group II flow port is axially aligned with one said fourth said Group II flow port, wherein each said second said Group II flow port is axially aligned with one said first said Group II flow port, and wherein said fourth said Group I flow port, said third said Group I flow port, and said second said Group I flow port are axially aligned.

51. The MEMS filter module claim 50, wherein said plurality of fourth said Group II flow ports are disposed about said fourth said Group I flow port, and wherein said plurality of second said Group II flow ports are disposed about said second said Group I flow port.

52. The MEMS filter module claim 50, wherein said plurality of fourth said Group II flow ports are disposed about said fourth said Group I flow port, and wherein said plurality of second said Group II flow ports are disposed about said second said Group I flow port.

53. The MEMS filter module of claim 1, wherein said plurality of layers comprises first, second, third, fourth, and fifth layers, wherein said fourth layer is located between said fifth layer and said third layer, wherein said third layer is located between said fourth layer and said second layer, wherein said second layer is located between said third layer and said first layer, wherein said at least one flow port for each of said first, second, third, fourth, and fifth layers is selected from the group consisting of at least one Group I flow port, at least one Group II flow port, or any combination thereof, wherein said MEMS filter module further comprises third and fourth filter traps within said first filtering section, wherein all flow through each of said third and fourth filter traps is in said second dimension, wherein each of said first, second, third, and fourth filter traps are disposed at different elevations within said stack, wherein each said Group I and Group II flow port has a smaller flow resistance than either of said first, second filter, third, and fourth traps, wherein said fifth layer comprises a fifth said Group I flow port and is devoid of any said Group II flow port, wherein said fourth layer comprises a plurality of fourth said Group II flow ports and a fourth said Group I flow port, wherein said third layer comprises a plurality of third said Group II flow ports and is devoid of any said Group I flow port, wherein said second layer comprises a plurality of second said Group II flow ports and a second said Group I flow port, wherein said first layer comprises a first said Group I flow port and is devoid of any said Group II flow port, wherein said fourth filter trap is disposed in a flow path between said fifth said Group I flow port and each of said fourth said Group II flow ports, wherein said third filter trap is disposed in a flow path between said fourth said Group I flow port and each of said third said Group II flow ports, wherein said second filter trap is disposed in a flow path between each of said third said Group II flow ports and said second said Group I flow port, wherein said first filter trap is disposed in a flow path between each of said second said Group II flow ports and said first said Group I flow port, and wherein all flow between said fifth said Group I flow port and said first said Group I flow port must pass through at least one of said third and fourth filter traps and also must pass through at least one of said first and said second filter traps.

54. The MEMS filter module claim 53, wherein each said fourth said Group II flow port is axially aligned with one said third said Group II flow port and with one said second said Group II flow port, and wherein said fifth said Group I flow port, said fourth said Group I flow port, said second said Group I flow port, and said first said Group I flow port are s axially aligned.

55. The MEMS filter module claim 54, wherein said plurality of fourth said Group II flow ports are disposed about said fourth said Group I flow port, and wherein said plurality of second said Group II flow ports are disposed about said second said Group I flow port.

56. The MEMS filter module claim 53, wherein said plurality of fourth said Group II flow ports are disposed about said fourth said Group I flow port, and wherein said plurality of second said Group II flow ports are disposed about said second said Group I flow port.

57. A MEMS filter module, comprising:
a first pair of fabrication levels comprising a first pair of structures that collectively define a first filter trap, wherein each member of said first pair of structures is in a different fabrication level of said first pair of fabrication levels; and
a second pair of fabrication levels comprising a second pair of structures that collectively define a second filter trap, wherein each member of said second pair of structures is in a different fabrication level of said second pair of fabrication levels.
